(19) United States
(12) Patent Application Publication
(10) Pub. No.: US 2026/0279510 A1
(43) Pub. Date: Sep. 17, 2026

(54) Large Chemistry Model-Based Drug Design with Supervised and Reinforcement Learning

(71) Applicant: Gavin Ye, Paramus, NJ (US)

(72) Inventor: Gavin Ye, Paramus, NJ (US)

(21) Appl. No.: 19/563,362

(22) Filed: Mar. 11, 2026

Related U.S. Application Data
(60) Provisional application No. 63/772,498, filed on Mar. 15, 2025.

(51) Int. Cl. G16C 20/50; G16C 20/70; G16C 20/90

(52) U.S. Cl. CPC G16C 20/50; G16C 20/70; G16C 20/90

(57) ABSTRACT

Disclosed is a system for designing drug-like candidate compounds using computational learning and molecular generation techniques. The system retrieves molecular structure data and associated drug-property information from a database, converts the molecular structures into sequential representations, and computes numerical chemical descriptors. A drug-property evaluation model is trained using the sequential representations and numerical descriptors to generate feedback scores indicative of predicted drug properties. A generative chemistry language model produces new candidate molecules, which are evaluated using the trained evaluation model. Based on the feedback scores, parameters of the generative chemistry language model are iteratively updated to generate subsequent candidate molecules with improved predicted properties. The iterative process continues until one or more stopping criteria are satisfied, enabling identification of candidate molecules suitable for further analysis or synthesis.

### CROSS-REFERENCE TO RELATED APPLICATIONS

This application claims the benefit of U.S. Provisional Application No. 63/772,498, filed on Mar. 15, 2025, the entire contents of which are incorporated herein by reference.

### FIELD OF THE INVENTION

The present disclosure generally relates to computational drug discovery and specifically to systems for designing drug-like candidate compounds using data-driven molecular modeling and iterative evaluation techniques.

### BACKGROUND OF THE INVENTION

The statements in the background of the invention are provided to assist with understanding the invention and its applications and uses, and may not constitute prior art.

The discovery and evaluation of chemical compounds for therapeutic use have long formed a part of pharmaceutical research and medicinal chemistry activities. Over time, growth in chemical databases, experimental datasets, and computational resources has resulted in increased reliance on data-centric approaches for analyzing molecular structures and associated biological outcomes. Research efforts in such areas often involve examination of relationships between molecular composition, structural arrangement, and observed behavior in biological environments. As chemical libraries expand in size and diversity, management of molecular information and interpretation of associated data has become progressively complex. Computational systems have therefore been applied to assist with storage, comparison, and assessment of molecular information, particularly in early stages of compound assessment where large numbers of chemical candidates may be reviewed prior to laboratory investigation.

Conventional systems operating in the area of molecular evaluation frequently rely on predetermined rules, static scoring schemes, or limited descriptor sets derived from chemical properties. Said systems often process molecular data in isolated stages, where molecular representation, property estimation, and candidate selection occur as separate activities. Such separation may result in repeated assessment cycles that lack adaptive refinement based on outcomes of prior evaluations. In many cases, molecular candidates are generated using fixed templates or constrained design spaces, which may limit exploration of broader chemical variations. Evaluation of candidate suitability is frequently based on single-property measures or narrow objective sets, which may fail to reflect combined considerations relevant to therapeutic use, manufacturability, or safety. As a result, large portions of generated candidate sets may be discarded at later stages, leading to inefficiencies in computational and experimental effort.

Further limitations arise when feedback from property assessment does not meaningfully influence subsequent stages of candidate generation. In such arrangements, generation processes may repeatedly produce structurally similar compounds without meaningful improvement across multiple evaluation dimensions. Manual intervention is often required to adjust selection thresholds, revise screening criteria, or redirect exploration toward alternative molecular patterns. Dependence on manual oversight may introduce variability in assessment outcomes and may slow progression through candidate pools. Additionally, conventional approaches may face difficulty in accommodating diverse data representations or integrating heterogeneous sources of molecular information within a unified evaluation process. Constraints related to toxicity, stability, similarity to known compounds, or feasibility of synthesis are often applied as post-processing filters rather than as integrated considerations during earlier assessment phases.

Accordingly, challenges persist in achieving systematic, adaptive, and data-driven exploration of chemical candidates using existing computational practices. Limitations associated with static evaluation strategies, fragmented processing stages, and limited feedback integration continue to affect efficiency and consistency in compound assessment workflows. As chemical datasets continue to increase in scale and complexity, approaches that rely heavily on predefined criteria or manual adjustment may encounter scalability constraints. There exists an ongoing need for approaches that support continuous refinement of candidate assessment using accumulated data, balanced consideration of multiple molecular characteristics, and structured management of large candidate populations. Addressing such challenges remains relevant for improving efficiency and reliability across compound discovery and evaluation activities without reliance on extensive manual oversight.

### BRIEF SUMMARY OF THE INVENTION

The present disclosure provides a system employing one or more processors and one or more memories for designing a drug-like candidate compound through computational processing of molecular data. Said system retrieves molecular training data from a database, said molecular training data comprising molecular structure information for a plurality of training molecules and drug-property matrices associated with said training molecules. Sequential molecular representations are generated for each training molecule, numerical chemical descriptors are computed, and a drug-property evaluation model is trained using said representations and descriptors. A generative chemistry language model generates candidate molecules, feedback scores are computed for said candidate molecules, and parameters of said generative chemistry language model are updated based on said feedback scores until satisfaction of a stopping criterion, thereby supporting iterative refinement of candidate molecules.

In an aspect, said drug-property matrices comprise experimental activity labels that are target-specific for at least one biological target. Said experimental activity labels represent measured interaction outcomes between training molecules and biological targets and may be expressed as binding affinity values, inhibition measurements, effectiveness measurements, dissociation measurements, or percentage-based activity values. Such experimental activity labels provide quantitative reference data supporting prediction of drug-related properties during evaluation of candidate molecules generated through computational processing.

In an embodiment, training of said drug-property evaluation model comprises training of a quantitative structure-property predictor that maps sequential molecular representations and numerical chemical descriptors to feedback scores. Said quantitative structure-property predictor establishes relationships between molecular characteristics and predicted drug-related properties derived from said drug-property matrices. Such training supports generation of feedback scores reflecting predicted behavior of candidate molecules relative to reference molecular data.

In an aspect, said numerical chemical descriptors comprise one or more categories selected from physicochemical descriptors, structural descriptors, topological descriptors, molecular fingerprints, fragment descriptors, or learned descriptor embeddings derived from molecular data. Said numerical chemical descriptors provide numerical representations of molecular characteristics that support evaluation of molecular suitability during training and scoring operations performed by said drug-property evaluation model.

In an embodiment, said sequential molecular representations further comprise additional molecular information selected from chemical identifier strings, molecular graph representations, fingerprint vectors, substructure presence vectors, pharmacophore feature vectors, or scaffold representations. Such molecular information supports expanded representation of molecular structure and relationships, thereby enhancing processing flexibility during training, evaluation, and generation of candidate molecules.

In an aspect, said feedback score comprises a multi-objective score combining potency-related components and developability-related components. Said developability-related components comprise selectivity factors, stability factors, novelty scores, synthesizability scores, similarity scores, absorption scores, distribution scores, metabolism scores, toxicity risk scores, solubility scores, permeability scores, or clearance scores. Said multi-objective score supports balanced assessment of candidate molecules across multiple evaluation dimensions.

In an embodiment, said multi-objective score is computed using weighted aggregation of objective-specific subscores and threshold-based filtering that rejects candidate molecules violating a predetermined constraint. Said weighted aggregation supports prioritization of selected evaluation dimensions, while said threshold-based filtering prevents progression of candidate molecules failing to satisfy defined acceptance limits.

In an aspect, said stopping criterion comprises identification of candidate molecules exceeding a predetermined feedback score threshold, satisfaction of multiple drug-property constraints, reaching of a maximum update count, convergence of feedback scores across candidate molecules, satisfaction of a diversity condition, or selection of a ranked candidate set satisfying a selection rule. Said stopping criterion governs termination of iterative refinement operations performed by said system.

In an embodiment, updating of parameters of said generative chemistry language model comprises application of a policy optimization algorithm selected from proximal policy optimization, guided reward policy optimization, or direct preference optimization. Said policy optimization algorithm adjusts parameter values governing candidate molecule generation based on feedback scores obtained during evaluation stages.

In an aspect, said parameter updating further applies divergence control relative to a reference policy using regularization to constrain deviation between an updated policy and said reference policy. Said divergence control supports stability of parameter updates across iterative cycles while permitting controlled adaptation based on feedback scores.

In an embodiment, said system validates candidate molecules using one or more external in-silico validation tools selected from molecular docking engines, simulation engines, absorption distribution metabolism excretion toxicity predictors, pharmacophore predictors, cheminformatics toolkits, database query tools, or literature search tools. Feedback scores associated with candidate molecules are modified based on results obtained from said external validation tools.

In an aspect, updating of parameters of said generative chemistry language model is performed based on modified feedback scores derived from said external validation, thereby supporting generation of refined candidate molecules reflecting outcomes of additional computational assessment processes.

In an embodiment, said system trains a target-specific generative chemistry language model using experimental activity labels associated with a respective biological target. Said target-specific generative chemistry language model supports generation of candidate molecules directed toward molecular interaction with said biological target.

In an aspect, said system applies penalties to feedback scores for candidate molecules violating predetermined constraints selected from toxicity limits, structural alert limits, similarity limits, synthetic feasibility limits, target-specificity limits, or selectivity limits relative to off-targets. Said penalties reduce ranking or acceptance likelihood of candidate molecules failing to satisfy such constraints.

In an embodiment, said system applies structural alert filters to candidate molecules, said structural alert filters comprising reactive group filters, toxicophore filters, mutagenicity alert filters, rule-based physicochemical filters, metal chelator filters, unstable moiety filters, strained ring filters, genotoxicity alert filters, or covalent-binding alert filters. Candidate molecules triggering a preset number of said filters are excluded from further consideration.

In an aspect, said system computes non-excluded feedback scores for candidate molecules that do not trigger exclusion by said structural alert filters. Said non-excluded feedback scores support continued evaluation and refinement of acceptable candidate molecules during iterative processing.

In an embodiment, a method is provided that performs retrieval of molecular training data, generation of sequential molecular representations, computation of numerical chemical descriptors, training of a drug-property evaluation model, generation of candidate molecules, computation of feedback scores, and iterative updating of parameters of a generative chemistry language model until satisfaction of a stopping criterion.

In an aspect, said method further computes a synthetic feasibility score for each candidate molecule and updates feedback scores in response to said synthetic feasibility score. Said synthetic feasibility score reflects practicality of laboratory synthesis associated with candidate molecules.

In an embodiment, said method further selects at least one candidate molecule for synthesis upon satisfaction of a synthetic feasibility score threshold. Said selection supports transition of candidate molecules from computational assessment to physical evaluation activities.

In an aspect, said system further updates at least one of said drug-property evaluation model or said generative chemistry language model based on experimental assay results obtained from synthesized candidate molecules. Said updating supports incorporation of experimental outcome data into subsequent computational processing.

In an embodiment, a non-transitory computer-readable storage medium stores program instructions that, when executed by a hardware processor, cause execution of operations comprising retrieval of molecular training data, generation of sequential molecular representations, computation of numerical chemical descriptors, training of a drug-property evaluation model, generation of candidate molecules, computation of feedback scores, and iterative updating of parameters of a generative chemistry language model until satisfaction of a stopping criterion.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein.

Embodiments of the present disclosure will now be described, by way of example only, with reference to the following diagrams.

FIG. 1 shows a drug-like candidate compound design system, in accordance with an embodiment of the present disclosure.

FIGS. 2A and 2B illustrate a molecular representation system demonstrating conversion of molecular structure information of a training molecule into a sequential molecular representation, in accordance with embodiments of the present disclosure.

FIG. 3 illustrates an exemplary diagram for processing performed by a drug-property evaluation model that receives a sequential molecular representation and a numerical chemical descriptor and produces an output indicative of at least one predicted drug property, in accordance with embodiments of the present disclosure.

FIG. 4 illustrates an exemplary diagram for interpretation of a molecular fingerprint descriptor used as a numerical chemical descriptor, in accordance with embodiments of the present disclosure.

FIG. 5 illustrates a policy optimization and divergence-controlled updating framework for a generative chemistry language model, in accordance with embodiments of the present disclosure.

FIG. 6 illustrates an exemplary diagram for training of a generative chemistry language model using sequential molecular representations, in accordance with embodiments of the present disclosure.

FIG. 7 illustrates a method for designing a drug-like candidate compound, in accordance with the embodiments of the present disclosure.

FIG. 8 illustrates exemplary steps for computation of an output value using a single-layer neural processing diagram used within a drug-property evaluation model, in accordance with embodiments of the present disclosure.

FIG. 9 illustrates exemplary steps for training, fine-tuning, evaluating, validating, testing, selecting, and deploying a machine learning model for a large chemistry model (LCM)-based drug design system, in accordance with embodiments of the present disclosure.

FIGS. 10A, 10B, and 10C illustrate exemplary diagrams for comparison of distributions of numerical chemical descriptors between dataset molecules and generated molecules, in accordance with embodiments of the present disclosure.

FIG. 11 illustrates an exemplary computing architecture for execution of a system to design a drug-like candidate compound, in accordance with embodiments of the present disclosure.

FIGS. 12A, 12B, and 12C show examples of integrated language-molecule computing architectures, in accordance with an embodiment of the present disclosure.

FIGS. 13A and 13B show examples of language-molecule model integration architectures using token-wise and sequence-wise molecular injection, in accordance with an embodiment of the present disclosure.

FIG. 14 shows an example of graph-based molecular encoding and atom-wise embedding integration with a large chemistry model, in accordance with an embodiment of the present disclosure.

FIGS. 15A and 15B show examples of a multi-specialist training, reinforcement learning, and self-distillation framework for a large chemistry model (LCM), in accordance with an embodiment of the present disclosure.

FIG. 16 shows an example of an iterative tool-augmented molecule validation and refinement workflow driven by an integrated LLM-LCM system, in accordance with an embodiment of the present disclosure.

FIG. 17 shows an example of a multi-step automated drug discovery workflow executed by an integrated LLM-LCM system, in accordance with an embodiment of the present disclosure.

### DETAILED DESCRIPTION OF THE INVENTION

The following detailed description illustrates embodiments of the present disclosure and ways in which they can be implemented. Although some modes of carrying out the present disclosure have been disclosed, those skilled in the art would recognize that other embodiments for carrying out or practicing the present disclosure are also possible.

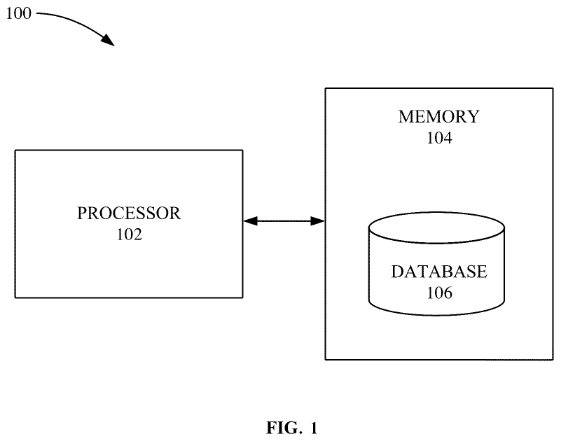

FIG. 1 shows a drug-like candidate compound design system 100, in accordance with an embodiment of the present disclosure. System 100 comprises one or more processors 102 and one or more memories 104 coupled to said one or more processors 102. Processor 102 executes program code stored in memory 104 to perform data processing operations associated with system 100. Processor 102 performs arithmetic operations and logical operations during execution of said program code. Processor 102 accesses data stored in memory 104 and writes data into memory 104 during execution of said program code. Processor 102 operates in coordination with memory 104 to support repeated execution of processing sequences under stored instructions.

In an embodiment, memory 104 stores program code, data structures, intermediate values, and parameter values accessed and modified by processor 102. Memory 104 comprises volatile storage and non-volatile storage arranged as random-access memory, cache memory, flash storage, solid-state storage, or magnetic storage. Memory 104 stores data used during execution of program code by processor 102 and stores data produced by processor 102. Memory 104 provides storage locations for persistent data and temporary data associated with operation of system 100.

In an aspect, processor 102 retrieves, from a database 106, a plurality of molecular training data comprising molecular structure information for a plurality of training molecules and a plurality of drug-property matrices corresponding to said plurality of training molecules. Each said drug-property matrix comprises one or more drug-property values associated with a respective training molecule. The term “drug-property matrix” as used throughout the present disclosure relates to a structured association between a training molecule and one or more drug-property values arranged as one or more vectors, tables, multi-field records, or label sets stored in association with said training molecule. Processor 102 stores said retrieved molecular training data into memory 104 as an indexed training dataset with record-level correspondence between said molecular structure information and said drug-property matrices. As used throughout the present disclosure, the term “drug-property matrices” refers to one or more machine-readable data structures that organize drug-related entities (including, without limitation, small-molecule candidates, known drugs, metabolites, fragments, or training molecules) in association with biological activity parameters into a matrix-form representation suitable for computational processing. In some embodiments, a drug-property matrix comprises a two-dimensional array in which a first dimension corresponds to drug identifiers (e.g., molecule IDs or structure-derived keys) and a second dimension corresponds to biological-activity fields, with each matrix entry storing a measured value, predicted value, class label, probability score, confidence score, or a missing-value indicator for a corresponding drug-activity pair. By way of non-limiting example, said biological activity parameters include target-specific potency or efficacy measures (e.g., IC₅₀, EC₅₀, Kᵢ, Kd), binding or inhibition percentages at specified concentrations, functional assay readouts, phenotypic response scores, selectivity indices across a panel of targets, pathway modulation scores, resistance or susceptibility scores, and/or cellular response endpoints (e.g., viability, proliferation, apoptosis induction, cytokine modulation) derived from in vitro, ex vivo, or in vivo studies. Accordingly, the drug-property matrices provide a standardized, model-ingestible representation of biological activity used for training, inference, candidate ranking, and multi-objective optimization of molecules against one or more biological targets or phenotypic endpoints.

In an aspect, processor 102 generates, for each training molecule in said plurality of training molecules, a sequential molecular representation selected from simplified molecular input line entry system (SMILES) strings and self-referencing embedded strings (SELFIES) to form a plurality of sequential molecular representations. Processor 102 acquires molecular structure information for the training molecule from memory 104 and converts said molecular structure information into a character sequence expressed as a SMILES string or a SELFIES. Processor 102 performs normalization operations on said molecular structure information prior to forming said character sequence, with normalization operations comprising charge normalization, aromaticity normalization, salt removal, and standardization of atom typing under prestored chemical rules. Processor 102 applies a consistent ordering rule during conversion so that identical molecular structure information yields said sequential molecular representation under repeated generation. Processor 102 stores said plurality of sequential molecular representations in memory 104 and associates each said sequential molecular representation with a corresponding training molecule identifier for subsequent training operations. As used throughout the present disclosure, the term “molecular structure information” refers to a machine-readable description of a molecule that specifies (i) an atom set with corresponding atom types (element identifiers) and (ii) a bond set identifying which atoms are connected and the associated bond orders, and may further include (iii) one or more structural attributes selected from formal charges, aromaticity designations, explicit and/or implicit hydrogen counts, stereochemical descriptors, isotopic labels, and salt/solvent components when present. In some embodiments, said molecular structure information is stored as a molecular graph, connection table, or other cheminformatics record from which a string encoding is deterministically derivable. By way of non-limiting example, an example molecular structure information (i.e., SMILES string) for arginine is “NC@@HC(═O)O”. In this example, stereochemical configuration is explicitly encoded at the α-carbon by the chiral marker “@@” in “[C@@H]”, such that an opposite stereoisomer may be represented by swapping the chirality marker (e.g., “[C@H]”) while maintaining the same atom connectivity. Further, charge state may be represented by including formal charge notation on one or more atoms using bracketed charged atoms, for example by representing a protonated amine terminus as “[NH3+]” and/or a protonated guanidinium functionality using an explicit positively charged nitrogen (e.g., “[NH2+]-C(N)N” as an illustrative fragment), thereby indicating that said molecular structure information includes a formal charge attribute that is preserved in the sequential molecular representation for machine processing and subsequent training operations. Processor 102 applies a consistent ordering rule during conversion so that identical molecular structure information yields said sequential molecular representation under repeated generation, and stores said plurality of sequential molecular representations in memory 104 with each said sequential molecular representation associated with a corresponding training molecule identifier for subsequent training operations.

In an example, molecular structure information for a training molecule representing C₈H₉NO₂ is stored in memory 104. Processor 102 reads said molecular structure information and converts said molecular structure information into a character sequence expressed as a SMILES string “CC(═O)NC1=CC═CC═C1O” or a SELFIES “[C][C][═O][N][C][Ring1][═C][C][═C][C][═C][C][═C][1][O]” to form said sequential molecular representation. During a later iteration, processor 102 reads said molecular structure information for said training molecule from memory 104 and applies said consistent ordering rule so that identical molecular structure information yields said sequential molecular representation under repeated generation.

In an aspect, processor 102 computes a numerical chemical descriptor associated with each training molecule in said plurality of training molecules to form a plurality of numerical chemical descriptors. Processor 102 computes descriptor vectors from said molecular structure information by executing descriptor computations that map sequential molecular representations into numeric values. Processor 102 computes said numerical chemical descriptor comprising molecular weight, log P, polar surface area, hydrogen bond donor count, hydrogen bond acceptor count, rotatable bond count, ring count, formal charge, and heavy atom count. Processor 102 computes topological descriptor values comprising connectivity indices, path-based indices, and adjacency-derived indices derived from a molecular graph representation stored in memory 104. Processor 102 also computes molecular fingerprint vectors as fixed-length bit vectors or fixed-length count vectors representing presence of substructures, fragments, or paths. Processor 102 stores numerical chemical descriptors in memory 104 as fixed-length vectors aligned with training molecule identifiers and aligned with sequential molecular representations for paired model input processing. The term “numerical chemical descriptor” as used throughout the present disclosure relates to a numeric value, numeric vector, fingerprint vector, fragment vector, or embedding vector derived from molecular structure information and representing one or more molecular characteristics for scoring and prediction.

In an example, molecular structure information for a training molecule representing C₈H₉NO₂ is stored in memory 104. Processor 102 reads said molecular structure information from memory 104 and computes said numerical chemical descriptor for said training molecule. Processor 102 computes numerical chemical descriptor values comprising a molecular weight value of about 151, a log P value of about 1.2, a polar surface area value of about 49, a hydrogen bond donor count of 2, a hydrogen bond acceptor count of 2, and a rotatable bond count of 2. Processor 102 arranges numerical chemical descriptor values into a numeric vector represented as [151, 1.2, 49, 2, 2, 2]. Processor 102 stores said numerical chemical descriptor in memory 104 as a fixed-length vector and associates said numerical chemical descriptor with a corresponding training molecule identifier.

In an aspect, processor 102 trains a drug-property evaluation model using the plurality of sequential molecular representations and the plurality of numerical chemical descriptors, wherein said drug-property evaluation model outputs a feedback score indicative of at least one predicted drug property associated with the plurality of drug-property matrices. Processor 102 constructs training samples by associating, for each training molecule identifier, the sequential molecular representation, the numerical chemical descriptor value, and the drug-property matrix label set retrieved from database 106. Processor 102 tokenizes sequential molecular representations into token sequences and maps token sequences into embedding vectors stored in memory 104 for training input formation. Processor 102 concatenates, fuses, or jointly processes said embedding vectors with numerical chemical descriptor values to form composite training inputs. Processor 102 trains model parameters by minimizing a loss value computed from differences between predicted property outputs and reference property values stored in drug-property matrices, with loss computation executed across batched training samples. Processor 102 generates the feedback score for each training sample during training by mapping predicted property outputs into a score representation, with score rules prestored in memory 104. The term “drug-property evaluation model” as used throughout the present disclosure relates to a learned mapping that receives at least one sequential molecular representation and at least one numerical chemical descriptor as input and produces predicted drug-property values used for derivation of the feedback score. The term “feedback score” as used throughout the present disclosure relates to a numeric value or a set of numeric values derived from one or more predicted drug-property values for a candidate molecule, where said feedback score enables ranking and comparison across candidate molecules.

In some embodiments, the drug-property evaluation model is generated as a family of model variants by varying one or more feature-design and training configurations. By way of non-limiting example, processor 102 may generate different model instances by varying (i) a number of numerical chemical descriptors provided as input features, (ii) a type, identity, and source of said numerical chemical descriptors (including inclusion or exclusion of selected descriptor categories, alternate descriptor calculation protocols, or alternate descriptor normalization schemes), and/or (iii) a count and arrangement of descriptors fused with the embedding vectors (e.g., early fusion, late fusion, gated fusion, or attention-based fusion). In some embodiments, processor 102 further modifies a statistical methodology used to determine efficacy or activity prediction quality, including selection of loss functions (e.g., regression loss for continuous potency values versus classification loss for active/inactive labels), weighting of assay endpoints to address imbalance or differing experimental confidence, application of calibration procedures to map raw outputs into probabilistic or ranked scores, and/or use of ensemble aggregation to reduce variance and improve robustness. In some embodiments, processor 102 generates multiple candidate models under said varying configurations and selects a preferred model based on comparative performance.

In some embodiments, each generated model is evaluated and validated using one or more statistical parameters computed over holdout datasets, cross-validation folds, or temporally separated evaluation sets. By way of non-limiting example, for continuous biological activity outputs (e.g., predicted IC₅₀ or K_{d}), processor 102 computes one or more regression performance metrics including mean absolute error (MAE), root mean squared error (RMSE), coefficient of determination (R²), and/or Pearson/Spearman correlation between predicted activity and reference activity stored in the drug-property matrices. For categorical outputs (e.g., active/inactive or responder/non-responder), processor 102 computes one or more classification metrics including area under the ROC curve (AUROC), area under the precision-recall curve (AUPRC), accuracy, precision, recall, F1-score, and/or Matthews correlation coefficient (MCC). In some embodiments, processor 102 additionally evaluates ranking quality of the feedback score using metrics such as top-k enrichment, hit rate, normalized discounted cumulative gain (NDCG), and/or Spearman rank correlation between score-based ranking and experimentally observed ranking. In some embodiments, processor 102 further computes reliability measures including calibration error (e.g., expected calibration error for probabilistic outputs), confidence intervals for selected metrics, and/or robustness measures under dataset shifts, and stores said evaluation outputs in memory 104 for selection, deployment, or iterative retraining of the drug-property evaluation model.

In an aspect, processor 102 generates, using a generative chemistry language model, at least one new candidate molecule to form a plurality of new candidate molecules (interchangeably referred as candidate molecule or candidate molecules). Processor 102 trains or loads parameter values for the generative chemistry language model using sequential molecular representations stored in memory 104 as training sequences. Processor 102 performs sequence generation by selecting a start token, repeatedly selecting next tokens from a probability distribution produced by said generative chemistry language model, and terminating generation upon reaching an end token or a maximum sequence length stored in memory 104. Processor 102 generates a plurality of candidate molecule sequences during an iteration by repeating sequence generation for a defined batch size stored in memory 104. Processor 102 performs validity checks on generated sequences by parsing generated sequences as SMILES strings or SELFIES and rejecting sequences that fail structural decoding. The term “generative chemistry language model” as used throughout the present disclosure relates to a generative model that produces sequential molecular representations as output sequences based on learned structure in training representations, where said output sequences correspond to chemically interpretable molecules under SMILES strings and SELFIES. Candidate molecule generation supports exploration of chemical sequence space, production of candidate pools with structural diversity under controlled sampling distributions, repeatable generation under stored sampling parameters in memory 104, and throughput scaling through batch generation executed by processor 102.

In an example, sequential molecular representations stored in memory 104 for training molecules are used as training sequences for the generative chemistry language model executed by processor 102. Processor 102 executes said generative chemistry language model and selects a start token. Processor 102 repeatedly selects next tokens from a probability distribution produced by said generative chemistry language model to generate a first SMILES string “CC1=CC═CC═C1O” and to generate a second SMILES string “CC(═O)NC1=CC═CC═CIF”. Processor 102 continues said sequence generation under stored sampling parameters in memory 104 until termination of each sequence occurs upon reaching an end token or a maximum sequence length. Processor 102 parses said first SMILES string and said second SMILES to form a plurality of new candidate molecules generated during a generation iteration.

In some embodiments, generation of the plurality of new candidate molecules is executed under multi-factor constraints and biasing rules stored in memory 104, such that the generative chemistry language model produces sequences that satisfy predetermined design objectives while maintaining chemical validity. By way of non-limiting example, processor 102 conditions sequence generation on one or more descriptor- or activity-driven guidance factors, including applying relative weights to selected descriptor types and/or predicted biological-activity outputs (e.g., weighting a potency-related objective more strongly than a selectivity-related objective) when sampling next tokens, reranking completed sequences, or accepting/rejecting generated candidates. In some embodiments, processor 102 further biases generation based on similarity to at least one reference molecule by computing a similarity metric between a generated candidate and a stored reference representation (e.g., a reference scaffold or reference active), and preferentially selecting candidates that fall within a target similarity range to enable exploitation around known actives while still permitting exploration of novel structures.

In some embodiments, processor 102 enforces or encourages generation consistent with known scaffolds, bioisosteric substitutions, and scaffold hopping. By way of non-limiting example, processor 102 may constrain generation to include a core scaffold stored in memory 104 while varying one or more substituent positions to yield an analog series or may replace a first substructure with a bioisosteric substructure to preserve biological interaction patterns while altering chemistry. For example, a phenyl substituent may be replaced with a heteroaryl ring, or a carboxylic acid substructure may be substituted with a tetrazole bioisostere, to maintain a similar interaction profile at a target site while modifying candidate characteristics. In some embodiments, processor 102 performs scaffold hopping by preserving a pharmacophore pattern inferred from activity data while changing the central core, thereby producing structurally distinct candidates that are predicted to retain target engagement.

In some embodiments, processor 102 reduces generation of candidates containing undesired substructures by applying toxicophore filtering and functional-group exclusion rules. By way of non-limiting example, processor 102 compares generated candidates against a prestored toxicophore set and rejects, penalizes, or downranks candidates containing one or more toxicological structural alerts (e.g., reactive electrophilic moieties or other high-risk functional groups defined in a stored alert library), thereby limiting candidate pools that are likely to exhibit adverse biological outcomes. In some embodiments, processor 102 also applies constraints to avoid substructures associated with known liabilities for the intended therapeutic area or intended administration route, as defined by prestored toxicology rules in memory 104.

In some embodiments, processor 102 incorporates target-binding-site requirements as generation guidance by biasing candidates toward structural features that support expected binding interactions at a biological target. By way of non-limiting example, where a target binding site includes a hydrophobic pocket, processor 102 may upweight generation of hydrophobic substituents predicted to occupy said pocket; where a binding site includes a hydrogen-bond donor or acceptor region, processor 102 may bias generation toward candidates bearing complementary hydrogen-bond acceptors or donors; and where an aromatic recognition region supports-T stacking, processor 102 may bias generation toward candidates having aromatic or heteroaromatic ring systems positioned to support said interaction. In some embodiments, processor 102 encodes such interaction requirements as interaction-feature constraints, pharmacophore constraints, or learned interaction priors derived from reference ligand-target data stored in database 106.

In some embodiments, processor 102 further guides candidate generation using structure-activity relationship (SAR) information derived from the drug-property matrices, such that modifications correlated with increased biological activity are preferentially explored while modifications correlated with decreased activity are suppressed. By way of non-limiting example, where the drug-property matrices indicate that introducing a hydrogen-bond acceptor at a first position of a scaffold increases potency against a target, processor 102 biases generation toward candidates incorporating an acceptor functionality at said position; and where the drug-property matrices indicate that adding a bulky substituent at a second position reduces activity due to steric clash, processor 102 downranks candidates incorporating said bulky substituent at said position. Accordingly, candidate molecule generation may be performed as a guided exploration of chemical sequence space that integrates similarity constraints, scaffold and bioisostere rules, toxicophore avoidance, target-interaction requirements, and SAR-derived priors to produce candidate pools optimized for the intended biological objective.

In one example, processor 102 may derive, from the drug-property matrices, one or more descriptor-activity relationships indicating that certain descriptor changes are positively correlated with a desired biological endpoint (e.g., increased potency or improved selectivity), while other descriptor changes are negatively correlated with said endpoint (e.g., reduced potency or reduced selectivity), and processor 102 may use such relationships to bias candidate molecule generation. In some embodiments, descriptor effects are learned per target and per assay, and the signs/magnitudes of such effects are stored as coefficients, feature importances, or monotonic constraints in memory 104. For example, where the drug-property matrices indicate that increased potency against a target is associated with an increased aromatic ring count and increased heteroaromatic ring fraction (consistent with stronger T-x stacking and optimized placement of hydrogen-bond acceptors in a binding site), processor 102 biases generation toward candidates that introduce or retain an aromatic/heteroaromatic moiety at a position corresponding to an aromatic interaction region in the binding site. In the same non-limiting example, the matrices may indicate that potency further increases when the hydrogen-bond acceptor count at a defined substituent region increases from a first range to a second range (e.g., adding one additional acceptor), and processor 102 therefore biases generation toward candidate molecules that include an additional acceptor functionality oriented toward a known hydrogen-bond donor region of the target. Conversely, if the matrices indicate that increasing rotatable bond count beyond a threshold correlates with reduced potency (e.g., due to an entropic penalty on binding), processor 102 downranks or rejects candidates having flexible linkers that increase rotatable bonds above said threshold, and instead biases generation toward more conformationally constrained linkers (e.g., ring closure, fused motifs, or reduced linker length) to improve predicted potency.

In some embodiments, selectivity is guided by descriptor-activity relationships derived from differences between on-target and off-target matrices. By way of non-limiting example, the matrices may indicate that improved selectivity is associated with an increased shape-complementarity proxy descriptor (e.g., increased fraction of sp³ centers or increased three-dimensionality index) that correlates with reduced off-target promiscuity, and processor 102 biases generation toward candidates that introduce saturated ring elements or sp³-rich substituents in regions not required for aromatic stacking, thereby increasing predicted selectivity while maintaining on-target interactions. Conversely, if the matrices indicate that increasing planarity-related descriptors (e.g., excessive aromatic density or high ring fusion count) correlates with reduced selectivity due to increased off-target binding across homologous targets, processor 102 penalizes candidates exhibiting said planarity-related descriptor increases and preferentially generates candidates having controlled aromatic content and increased three-dimensional features.

In some embodiments, processor 102 uses these learned relationships to generate candidates by applying explicit “increase/decrease” constraints on descriptors during sampling and post-generation filtering. By way of non-limiting example, processor 102 may generate a candidate series around a reference scaffold by (i) introducing a heteroaromatic ring nitrogen or a carbonyl group to increase a hydrogen-bond acceptor descriptor correlated with higher potency, (ii) adding a constrained ring or shortening a linker to reduce rotatable bond count correlated with higher potency, (iii) introducing an sp³-rich substituent to improve a three-dimensionality descriptor correlated with higher selectivity, and (iv) rejecting candidates that increase planarity-related descriptors beyond a selectivity threshold. Accordingly, candidate molecule preparation is executed as a descriptor-guided design process in which descriptors correlated with increased activity or selectivity are promoted and descriptors correlated with reduced potency or reduced selectivity are suppressed, thereby enriching the generated candidate pool for the intended biological performance profile.

In some embodiments, processor 102 generates candidate molecules by introducing a chain and/or altering chain length at one or more attachment points to enable systematic exploration of a binding pocket, such that a substituent is extended, shortened, or repositioned to probe pocket depth, subpockets, and vector directions associated with activity and selectivity. By way of non-limiting example, processor 102 may vary an alkyl, heteroalkyl, or linker segment by one or more atoms to shift a terminal functional group toward a distal hydrogen-bond region or deeper hydrophobic cavity and may optionally introduce branching to access a lateral subpocket while preserving a core pharmacophore. In some embodiments, processor 102 further constrains a scaffold by selecting more rigid linkers, introducing ring closure, or reducing rotatable bonds to limit conformational freedom, thereby reducing variation in binding mode and promoting a consistent pose that preserves key interaction geometry across an analog series. In some embodiments, processor 102 additionally performs stereochemistry analysis by generating stereoisomeric variants (e.g., chiral center inversion or stereodefined substituent placement) and evaluating predicted biological activity and selectivity for each stereoisomer, such that a preferred stereochemical configuration is selected when said configuration improves binding complementarity, reduces steric clash, or enhances directional interactions (e.g., hydrogen bonding or x-x alignment) relative to an alternate configuration.

In an aspect, processor 102 computes, by the drug-property evaluation model, the feedback score for each candidate molecule in the plurality of candidate molecules to form a plurality of feedback scores. Processor 102 converts each generated candidate molecule sequence into model input form by tokenization and embedding generation consistent with training preprocessing stored in memory 104. Processor 102 computes, for each candidate molecule, predicted property outputs using the trained drug-property evaluation model and computing the feedback score derived from said predicted property outputs using score rules stored in memory 104. Processor 102 stores candidate molecule identifiers, candidate molecule sequences, predicted property outputs, and feedback scores as linked records in memory 104 for ranking and update processing. Processor 102 ranks candidate molecules within a batch by ordering candidate molecules according to feedback score and stores ranked candidate lists in memory 104. Feedback score computation supports ranking of candidate molecules under a single scoring framework derived from drug-property matrices, rejection of structurally invalid candidate molecule sequences prior to model scoring through SMILES string or SELFIES string parsing, monitoring of score distributions across iterations using stored batch score statistics in memory 104, and selection of high-scoring candidate molecules for subsequent parameter update influence.

In an aspect, processor 102 updates one or more parameters of the generative chemistry language model based on the plurality of feedback scores, wherein said updating causes said generative chemistry language model to generate a subsequent candidate molecule to form a plurality of subsequent candidate molecules having an improved feedback score relative to the plurality of candidate molecules, and wherein said updating and said generation continue until at least one stopping criterion is satisfied. Processor 102 assigns a reward value to each subsequent candidate molecule sequence based on the feedback score and forms a reward-weighted training signal stored in memory 104. Processor 102 updates parameter values of the generative chemistry language model by increasing likelihood of token sequences associated with higher reward values and reducing likelihood of token sequences associated with lower reward values, with parameter gradients computed across a batch of candidate molecules. Processor 102 stores updated parameter values in memory 104 as an updated parameter set indexed by iteration count and generates subsequent candidate molecules using said updated parameter set. Processor 102 evaluates the stopping criterion by comparing best feedback score values, average feedback score values, score variance values, diversity metrics, and iteration count values against termination rules prestored in memory 104. The term “stopping criterion” as used throughout the present disclosure relates to one or more termination rules stored in memory 104 and evaluated by processor 102, where said termination rules determine when iterative update cycles end based on, score thresholds, iteration limits, score convergence conditions, diversity conditions, or ranked selection.

The one or more parameter values may comprise, by way of non-limiting example, model weight matrices, token embedding vectors, attention and projection parameters, output-layer parameters that define token likelihoods, and/or one or more generation-control parameters that influence sampling behavior. In one implementation, processor 102 assigns, for each candidate molecule sequence generated in an iteration, a respective reward value derived from the corresponding feedback score and forms a reward-weighted training signal that is applied to modify said one or more parameter values. Processor 102 may update said one or more parameter values according to one or more update rules stored in memory 104, wherein said update rules specify conditions under which parameter modification is applied, limited, delayed, or reverted. Exemplary update conditions selected from satisfaction of a minimum batch validity condition (e.g., at least a threshold fraction of generated sequences correspond to chemically valid structures), satisfaction of a minimum improvement condition (e.g., a best score value, an average score value, or a percentile score value exceeds a prior iteration value or a moving baseline value by at least a prestored margin), satisfaction of a diversity condition (e.g., a scaffold diversity metric, novelty metric, or pairwise similarity statistic remains above a prestored diversity floor), and satisfaction of a stability condition limiting distributional drift (e.g., a constraint on change in token probability distribution between successive parameter sets). In this manner, processor 102 increases likelihood of token sequences associated with higher reward values and reduces likelihood of token sequences associated with lower reward values while maintaining controlled exploration and preventing premature mode collapse, and stores updated parameter values in memory 104 as an indexed parameter set associated with a corresponding iteration count.

In a further aspect, processor 102 evaluates one or more stopping criteria to determine when the iterative update and generation cycles terminate, wherein the stopping criteria comprise one or more termination rules prestored in memory 104. Exemplary stopping criteria selected from: (i) a score attainment rule in which a best feedback score value and/or an average feedback score value meets or exceeds a prestored target threshold for at least a prestored number of consecutive iterations; (ii) a convergence rule in which improvement in one or more score statistics (best, average, or percentile) is less than a prestored tolerance for at least a prestored number of iterations, optionally in combination with a score variance rule indicating stabilization of the score distribution; (iii) a diversity rule in which one or more diversity metrics falls below a prestored minimum diversity threshold for at least a prestored number of iterations; (iv) an iteration or resource limit rule in which an iteration count, compute budget, or elapsed time reaches a prestored limit; and/or (v) a ranked selection rule in which at least a prestored number of candidate molecules satisfying one or more property constraints (e.g., potency, selectivity, and/or developability constraints) has been identified. Processor 102 terminates the iterative update cycle upon satisfaction of any one termination rule or a defined combination of termination rules, and outputs, stores, and/or ranks one or more candidate molecules associated with the best feedback score values and/or satisfying the property constraints.

In an embodiment, the plurality of drug-property matrices stored in database 106 may comprise at least one experimental activity label that is target-specific for at least one biological target and said experimental activity label is selected from binding affinity, half maximal inhibitory concentration, half maximal effective concentration, inhibition constant, dissociation constant, and inhibition percentage. Processor 102 may retrieve said drug-property matrices from database 106 and may store said experimental activity label in memory 104 in association with training molecule identifiers and biological target identifiers. Said biological target may correspond to a protein (for example, BRAF kinase protein, etc.), receptor (for example, EGFR (Epidermal Growth Factor Receptor), etc.), enzyme (for example, Cyclooxygenase-2 (COX-2), etc.), ion channel (e.g., Ca-channel), nucleic acid sequence (for example, SARS-CoV-2 RNA-dependent RNA polymerase gene sequence, etc.), or molecular complex (for example, PD-1/PD-L1 protein-protein complex, etc.) associated with a disease pathway. Said experimental activity label may be stored as a numeric value, a bounded range, or a normalized value aligned with an assay identifier and a target identifier, with units and scaling recorded as part of database 106.

In an embodiment, target-specific labeling may enable separation of molecular training data by biological target so that score generation performed by processor 102 aligns with target context rather than general chemical similarity. Binding affinity labels may represent interaction strength, IC₅₀ labels may represent inhibition level under defined assay conditions, EC₅₀ labels may represent response level under defined stimulation conditions, and Kᵢ or K_{d} labels may represent kinetic or equilibrium properties derived from measurement or fitting. Inhibition percentage labels may represent categorical or continuous readouts under a defined concentration and time condition. Such target-specific labeling may reduce mixing of unrelated property signals across targets in memory 104, improve relevance of feedback score computed by processor 102 for a selected biological target, enable more stable ranking of candidate molecules stored in memory 104 under a consistent assay definition, and support screening workflows that prioritize candidate molecules associated with stronger drug-property matrices stored in database 106.

In an embodiment, preparation of said experimental activity label may employ assay normalization, outlier handling, replicate aggregation, and label transformation so that numeric distributions remain suitable for supervised learning performed by processor 102. Label transformation may use log scaling for concentration-based labels, percentile scaling for cross-assay harmonization, or clipping for extreme values recorded as censoring thresholds. Database 106 may store confidence indicators or measurement qualifiers that influence training sample weighting applied by processor 102, and memory 104 may store such weighting values for repeated training cycles.

In another embodiment, training of the drug-property evaluation model performed by processor 102 may comprise training of a quantitative structure-property relationship (QSPR) predictor that maps the plurality of sequential molecular representations stored in memory 104 and the plurality of numerical chemical descriptors stored in memory 104 to the plurality of feedback scores generated by processor 102. Said QSPR predictor may receive sequential molecular representations as token sequences and numerical chemical descriptors as fixed-length vectors aligned to corresponding training molecules retrieved from database 106. Said QSPR predictor may output one feedback score per training molecule, or said predictor may output a set of predicted property values that are converted into one feedback score under score rules stored in memory 104.

In another embodiment, mapping behavior of said QSPR predictor may represent learned associations between molecular patterns in sequential molecular representations and drug-related property trends captured by numerical chemical descriptors. Sequential molecular representations may carry information about atom ordering, branching, ring closures, and stereochemical indicators under a selected encoding, while numerical chemical descriptors may carry information about size, polarity, topology, fragment presence, or other computed characteristics. Joint processing of such inputs by processor 102 may enable improved prediction for molecules that share descriptor similarity but differ in sequence patterns and may enable improved prediction for molecules that share sequence motifs but differ in descriptor magnitudes. Such mapping may improve feedback score sensitivity to combined structural and physicochemical variation and reduce reliance on any single representation type stored in memory 104.

In another embodiment, training of said QSPR predictor may employ supervised objectives that penalize deviation between predicted property outputs and reference values stored in drug-property matrices in database 106, with sample weighting applied based on label confidence or target relevance stored in database 106. Training may employ batch training with shuffling controls so that training molecule exposure remains balanced across property ranges, and training may employ early stopping based on validation error trends stored in memory 104. Training may also employ calibration procedures that align predicted score distributions with reference score distributions so that feedback score thresholds stored in memory 104 remain meaningful during iterative candidate generation. The training may improve stability of feedback score distribution across iterative update cycles executed by processor 102. Aforesaid training may reduce drift in feedback score interpretation between training and deployment and may enable more reliable comparison of candidate molecules stored in memory 104 across iterations.

In an embodiment, the plurality of numerical chemical descriptors computed by processor 102 and stored in memory 104 may comprise at least one selected from a physicochemical descriptor, a structural descriptor, a topological descriptor, a molecular fingerprint, a fragment descriptor, and a learned descriptor embedding. Said physicochemical descriptor may represent molecular weight, log P, polar surface area, hydrogen bond donor count, hydrogen bond acceptor count, rotatable bond count, ring count, formal charge, or heavy atom count. Said structural descriptor may represent substructure counts, functional group counts, or ring system categories, and said topological descriptor may represent connectivity indices, path-based indices, or adjacency-derived indices derived from a molecular graph representation derived from molecular structure information retrieved from database 106.

In an embodiment, the molecular fingerprint stored in memory 104 may represent a fixed-length bit vector or count vector indicating presence of substructures, fragments, or paths under a selected fingerprint scheme, and the fragment descriptor may represent fragment frequency values derived from decomposition of a molecular structure. The learned descriptor embedding may represent a vector produced by a learned encoder executed by processor 102 that processes molecular structure information from database 106 or sequential molecular representations stored in memory 104 and outputs an embedding capturing latent chemical relationships. Storage of said numerical chemical descriptors in a consistent vector format may enable batch training and batch scoring operations executed by processor 102. Such descriptor usage may improve capture of chemical similarity beyond string-level similarity and may enable stable model input dimensionality across different molecules stored in memory 104.

In an embodiment, descriptor computation executed by processor 102 may apply standardization rules prestored in memory 104 so that descriptor scale remains consistent across training molecules and candidate molecules. Standardization may apply mean-variance normalization, min-max scaling, log transforms for skewed descriptors, and missing value imputation under defined standardization rules. Descriptor computation may also apply feature selection rules that remove constant descriptors or highly correlated descriptors so that training remains stable and efficient.

In another embodiment, the sequential molecular representation generated by processor 102 and stored in memory 104 further comprises information selected from an international chemical identifier (InChI) string, a molecular graph representation, a fingerprint vector, a substructure presence vector, a pharmacophore feature vector, and a scaffold representation. Said InChI string may provide a standardized text identifier for a molecular structure derived from said molecular structure information retrieved from database 106 and said molecular graph representation may provide node-edge data representing atoms and bonds derived from said molecular structure information. Said fingerprint vector and said substructure presence vector may provide sparse or dense indicators stored in memory 104 that complement sequential molecular representations expressed under SMILES strings or SELFIES.

In another embodiment, a pharmacophore feature vector stored in memory 104 may represent arrangements of chemical features associated with interaction potential, such as hydrogen bond donor features, hydrogen bond acceptor features, hydrophobic features, aromatic features, or charged features. A scaffold representation stored in memory 104 may represent a core framework derived by removing substituents under a defined scaffold extraction rule executed by processor 102. Combining sequential molecular representations with such additional information may enable representation of both detailed substituent patterns and core frameworks.

In another embodiment, integration of said additional information may employ parallel input streams processed by a scoring model executed by processor 102, with output fusion performed by concatenation, attention-based fusion, or weighted aggregation stored in memory 104. The InChI string may enable de-duplication checks during candidate generation and evaluation and said scaffold representation may enable diversity constraints during iterative selection. Said pharmacophore feature vector may enable prioritization of candidate molecules matching a desired feature pattern stored as a reference pattern in memory 104. Such integration may reduce duplicate candidate generation across batches stored in memory 104 and enable enforcement of diversity conditions without modifying score rules.

In an embodiment, the feedback score computed by processor 102 and stored in memory 104 comprises a multi-objective score selected from a potency component and a developability component, where the developability component comprises at least one selected from a selectivity factor, a stability factor, a novelty score, a synthesizability score, a similarity score, a potency score, an absorption score, a distribution score, a metabolism score, a toxicity risk score, a solubility score, a permeability score, and a clearance score. Said potency component may reflect predicted target interaction strength derived from labels stored in drug-property matrices in database 106 and said developability component may reflect predicted suitability for downstream development stages under multiple criteria stored as score rules in memory 104.

In an embodiment, said multi-objective score may be formed by combining objective-specific subscores so that candidate molecules with strong potency but poor developability receive reduced overall ranking relative to candidate molecules with balanced properties. Said selectivity factor may represent predicted difference between on-target interaction and off-target interaction, said stability factor may represent predicted chemical or metabolic stability, and said toxicity risk score may represent predicted likelihood of adverse effects. Said synthesizability score may represent predicted synthetic accessibility under known score rules stored in memory 104 and said similarity score may represent distance from known molecules under fingerprints or scaffolds derived from database 106. Such multi-objective scoring supports prioritization of candidate molecules that balance potency with safety-related properties, reduces selection of candidate molecules exhibiting single-property dominance with multi-property weakness, maintains ranking stability under shifting candidate pool composition across iterations stored in memory 104, and guides iterative updates toward generation of balanced candidate molecules rather than extreme candidate molecules.

In an embodiment, weighting rules for objective-specific subscores may be stored in memory 104 and adjusted across runs, where higher weights may be assigned to toxicity risk or stability for safety-prioritized workflows, and higher weights may be assigned to potency for efficacy-prioritized workflows. Threshold may be applied to reject candidate molecules when one or more subscores exceed unacceptable limits stored in memory 104, such as toxicity risk above a toxicity threshold or solubility below a solubility threshold. Use of such weighting and thresholding may align iterative updates with defined development priorities and reduce wasted scoring cycles on candidate molecules outside acceptance boundaries.

In an embodiment, the multi-objective score may be computed using a weighted aggregation of a plurality of objective-specific subscores and one or more threshold-based filters that reject the candidate molecule that violates a first predetermined constraint. Said objective-specific subscores may correspond to potency-related scoring and developability-related scoring stored in memory 104, and said weighted aggregation may apply weight values stored in memory 104 to generate an aggregated score value. Said threshold-based filters may evaluate each objective-specific subscore against one or more threshold values stored in memory 104. The weighted aggregation may enable emphasis on selected scoring components during feedback score computation by processor 102 and may enable removal of candidate molecules that fail threshold values prior to parameter updating by processor 102.

In an embodiment, said weighted aggregation may apply fixed weight values for a run, or said weighted aggregation may apply weight schedules that vary across iteration count values stored in memory 104. Weight schedules may assign larger weights to potency during early iterations and may assign larger weights to developability during later iterations, based on prestored iteration stage rules. Said threshold-based filters may operate as hard filters that reject candidate molecules upon violation of a single threshold, or said threshold-based filters may operate as multi-threshold filters that reject candidate molecules upon violation of a preset number of thresholds. Such weight scheduling supports staged refinement of candidate molecule pools during iterative processing by processor 102, with such hard filtering and such multi-threshold filtering supporting faster removal of candidate molecules associated with unacceptable property predictions and consistent candidate molecule quality control prior to parameter update operations stored in memory 104.

In an embodiment, said first predetermined constraint may relate to toxicity-risk limits, solubility limits, similarity limits, stability limits, clearance limits, or permeability limits, where each limit may correspond to a threshold stored in memory 104. Said threshold-based filters may evaluate a predicted subscore output generated by the drug-property evaluation model executed by processor 102 and may output a pass result or a reject result stored in memory 104. Candidate molecules associated with a rejected result may be removed from ranked sets prior to generation of subsequent candidate molecules. Such constraint evaluation supports reduction of candidate molecules with predicted undesirable properties in ranked sets stored in memory 104, reduces update influence from rejected candidate molecules during parameter updating by processor 102, and maintains separation between acceptable candidate molecules and unacceptable candidate molecules within each batch.

In another embodiment, a stopping criterion may comprise identification of at least one subsequent candidate molecule with an associated feedback score exceeding a predetermined threshold, identification of at least one subsequent candidate molecule satisfying a plurality of drug-property constraints, reaching of a maximum number of update iterations, convergence of feedback scores across candidate molecules, meeting of a diversity criterion across a subset of candidate molecules, or selection of a ranked set of candidate molecules satisfying a selection rule. Said predetermined threshold, said maximum number, said convergence limits, said diversity limits, and said selection rule may be stored in memory 104 and evaluated by processor 102 during iterative updating.

In another embodiment, convergence of feedback scores may be evaluated by processor 102 using batch statistics stored in memory 104, where said batch statistics may comprise average feedback score values, variance values, percentile values, or moving-window trend values. Convergence may be detected when change in a batch statistic remains below a convergence threshold for a defined number of iterations stored in memory 104. The diversity criterion may be evaluated using similarity measures derived from sequential molecular representations stored in memory 104 or derived from numerical chemical descriptors stored in memory 104. The convergence evaluation may enable detection of diminishing score improvement across iterations and may enable termination decisions that limit repeated generation of similar candidate molecules. The diversity evaluation may enable retention of structurally varied candidate molecules within selected subsets and may enable avoidance of subsequent candidate molecule collapse toward a narrow chemical pattern during iterative updating.

In another embodiment, satisfaction of a plurality of drug-property constraints may be determined when a subsequent candidate molecule meets multiple thresholds stored in memory 104, where each threshold rule may correspond to a different predicted property dimension output by the drug-property evaluation model executed by processor 102. Selection of the ranked set may be performed by ordering candidate molecules using feedback scores stored in memory 104 and selecting a top portion based on a selection rule stored in memory 104. Said selection rule may reference top-K selection, percentile selection, or constraint-qualified selection. Such multi-constraint satisfaction may enable selection of candidate molecules with balanced predicted properties rather than single-dimension strength. The ranked-set selection may enable downstream processing focus on a manageable subset size stored in memory 104. The rule-based selection may enable repeatable selection behavior across runs under stored rule values. The stopping mechanisms may enable generation of candidate molecule outputs that remain aligned with stored acceptance thresholds.

In an embodiment, updating of one or more parameters of the generative chemistry language model may comprise applying a policy optimization algorithm selected from proximal policy optimization (PPO), guided reward policy optimization (GRPO), and direct preference optimization (DPO). Said policy optimization algorithm may operate on sequences representing candidate molecules produced by the generative chemistry language model executed by processor 102 and may use feedback scores stored in memory 104 as reward signals or preference signals. Said policy optimization algorithm may adjust parameter values stored in memory 104 so that generation probability for higher-scoring sequences may increase relative to lower-scoring sequences. The policy optimization may allow feedback-driven adjustment of generative parameter values stored in memory 104 and enable repeated improvement of candidate molecule score distributions across iterations executed by processor 102.

In an embodiment, said PPO may operate by computing a ratio between likelihood values under an updated policy and likelihood values under a prior policy, with ratio clipping applied using clip limits stored in memory 104. The GRPO may operate by shaping reward signals using reward baselines, reward normalization, or reward shaping rules prestored in memory 104 so that update gradients may remain stable across batches. The DPO may operate using pairwise preference labels derived from feedback scores, where higher feedback score sequences may be treated as preferred relative to lower feedback score sequences. Such ratio clipping may reduce instability during parameter updating across iterations. Such reward shaping reduces variance in reward gradients computed by processor 102. The preference-based training may enable learning from relative ranking information rather than absolute score magnitude.

In an embodiment, policy optimization processing may use batch candidate pools stored in memory 104, where each candidate pool may comprise candidate molecule sequences, feedback scores, and auxiliary statistics such as score percentiles or diversity measures. Policy optimization processing may compute gradients using said batch candidate pools and may apply gradient accumulation across multiple batches based on accumulation rules prestored in memory 104. Policy optimization processing may store parameter checkpoints in memory 104 per iteration for rollback or comparison.

In another embodiment, parameter updating may further apply Kullback-Leibler regularization relative to a reference policy to constrain divergence between an updated policy and said reference policy. Said reference policy may correspond to a baseline parameter set stored in memory 104, where said baseline parameter set may correspond to a pre-update generative parameter set or a fixed reference parameter set. Said Kullback-Leibler regularization may compute divergence values between token probability distributions under an updated policy and token probability distributions under said reference policy across candidate sequences. Such divergence control supports limitation of parameter drift across iterative updates stored in memory 104, retention of chemical syntax validity learned in baseline training sequences stored in memory 104, and reduction of abrupt changes in candidate molecule distribution across iterations executed by processor 102.

In another embodiment, said Kullback-Leibler regularization may be applied as an added penalty term within a training objective, where penalty strength values may be stored in memory 104 and may be set per run or per iteration schedule. Penalty strength values may be increased when divergence values exceed a divergence threshold stored in memory 104, and penalty strength values may be reduced when divergence values remain below said divergence threshold. Divergence thresholds may be computed using average divergence across a batch, percentile divergence across a batch, or moving-window divergence trends stored in memory 104. Such penalty scheduling may enable adaptive control of divergence based on observed divergence statistics.

In another embodiment, reference-policy divergence control may operate alongside reward maximization so that parameter updating may balance score improvement and distribution stability. When feedback scores are associated with large divergence, penalty strength values may increase to reduce divergence, and when feedback scores are associated with small divergence, penalty strength values may decrease to allow stronger exploitation of scoring gradients. Reference policy selection may use an immediately preceding policy stored in memory 104 or a fixed baseline policy stored in memory 104. Such balancing may enable controlled improvement of feedback scores while maintaining stable decoding behavior.

In an embodiment, one or more processors 102 may validate each candidate molecule or each subsequent candidate molecule using one or more external in-silico validation tools, where said external in-silico validation tools comprise at least one selected from a molecular docking engine, a molecular dynamics or simulation engine, an absorption distribution metabolism excretion toxicity (ADMET) predictor, a pharmacophore predictor, a cheminformatics toolkit, a database query tool, and a literature search tool, and processor 102 may modify the feedback score for each candidate molecule based on such validation. Said external in-silico validation tools may operate on molecular structures derived from sequential molecular representations stored in memory 104, and results from said external in-silico validation tools may be stored in memory 104 as validation outputs aligned to candidate molecule identifiers. Such validation supports additional evaluation beyond model-predicted scores derived from training labels stored in database 106, detection of candidate molecules with poor simulated interaction behavior prior to parameter updating, and adjustment of feedback scores to reflect multi-source evidence derived from external computational tools.

In an embodiment, said molecular docking engine may output docking scores, pose stability metrics, or interaction fingerprints that may be converted into validation subscores stored in memory 104. Said simulation engine may output trajectory stability metrics, solvation-related measures, or conformational stability measures that may influence feedback score rules stored in memory 104. Said ADMET predictor may output predicted absorption values, predicted clearance values, predicted metabolism flags, or predicted toxicity-risk indicators that may be used as penalty terms applied to feedback scores. The conversion of validation outputs may allow feedback score refinement based on structural interaction estimates. Such simulation-informed scoring may enable rejection of candidate molecules with unstable binding pose behavior. Said ADMET-informed penalty application may enable lower ranking for candidate molecules with predicted unfavorable disposition properties.

In an embodiment, said cheminformatics toolkit may compute additional descriptors, perform structure standardization checks, detect reactive group alerts, or compute similarity measures relative to molecules stored in database 106, and processor 102 may use such outputs to adjust feedback scores stored in memory 104. Said database query tool may retrieve known activity records or known safety annotations from database 106 or from an external data store, and processor 102 may apply score adjustments based on presence of matched records. Said literature search tool may retrieve references associated with structural motifs, and processor 102 may apply score adjustments when said references indicate undesirable motifs. Such toolkit integration may allow feedback score adjustment using standardized cheminformatics computations. The database-linked adjustment may enable avoidance of candidate molecules closely matching undesirable known records. The literature-linked adjustment may enable early screening for motif-associated risk signals.

In an embodiment, one or more processors 102 update one or more parameters of the generative chemistry language model based on a modified feedback score and said modified feedback score may be derived from the feedback score adjusted using validation outputs stored in memory 104. Said modified feedback score may be associated with each candidate molecule represented as the sequential molecular representation stored in memory 104 and said modified feedback score may reflect a combination of predicted property outputs and validation-derived adjustments. Said modified feedback score may be stored in memory 104 together with candidate molecule identifiers and ranking data. Such modified feedback score storage supports traceable linking between validation outputs and parameter update inputs and supports comparison between unmodified feedback scores versus modified feedback scores across candidate molecule pools.

In an embodiment, parameter updating based on said modified feedback score may shift probability mass of token sequences toward candidate molecule sequences associated with improved modified feedback scores and may shift probability mass away from candidate molecule sequences associated with reduced modified feedback scores. Such updating may use batch candidate pools stored in memory 104, where each candidate pool may carry candidate sequences and associated modified feedback scores. Subsequent candidate molecule generation under updated parameter values may yield refined candidate molecules that trend toward higher modified feedback scores under the same score rules stored in memory 104.

In an embodiment, modification of the feedback score may apply additive adjustments, multiplicative adjustments, penalty adjustments, or threshold-triggered adjustments stored as score rules in memory 104. A docking-derived adjustment may reduce feedback score when a docking score exceeds a stored threshold, and an ADMET-derived adjustment may reduce the feedback score when a predicted toxicity risk exceeds a stored threshold. A simulation-derived adjustment may reduce the feedback score when stability metrics fall below a stored threshold. Such structured adjustment may enable consistent application of modified feedback scoring across candidate pools and may allow tunable emphasis between predicted properties and validation outputs.

In another embodiment, one or more processors 102 train a target-specific specialist generative chemistry language model using a plurality of target-specific experimental activity labels, where said target-specific experimental activity labels may be stored in drug-property matrices in database 106. Said target-specific specialist generative chemistry language model may be trained for a respective biological target so that generation behavior aligns with molecular patterns associated with activity labels for said respective biological target. Memory 104 may store biological target identifiers, target-specific training subsets, and target-specific parameter sets for said target-specific specialist generative chemistry language model.

In another embodiment, training for a respective biological target may use sequential molecular representations aligned to said respective biological target and may use activity labels such as binding affinity, IC₅₀, EC₅₀, Kᵢ, K_{d}, or inhibition percentage aligned to said respective biological target. Said target-specific specialist generative chemistry language model may produce candidate molecules that cluster around sequence motifs and scaffolds correlated with target-specific activity labels stored in database 106. Said target-specific specialist generative chemistry language model may be used within iterative updating so that candidate molecule generation and candidate molecule scoring remain aligned with a selected biological target context. The target-focused generation may reduce mixing of unrelated biological targets during candidate molecule generation and may enable higher density of candidate molecules associated with a selected biological target within a candidate pool.

In another embodiment, conditioning of said target-specific specialist generative chemistry language model may use target tokens, target identifier embeddings, or target-conditioned prompts stored in memory 104, where said conditioning information may guide sequence generation toward target-specific chemical patterns. Target-specific training may use balanced sampling across activity ranges so that generation does not collapse toward a narrow score band, and target-specific training may use diversity constraints derived from fingerprints or scaffolds stored in memory 104. Such conditioning may enable rapid retargeting of generation between biological targets through target token selection.

In an embodiment, one or more processors 102 apply a penalty to the feedback score for each candidate molecule that violates a second predetermined constraint selected from a toxicity constraint, a structural alert constraint, a similarity constraint, a synthetic feasibility constraint, a target-specificity constraint relative to a biological target, and a selectivity constraint relative to one or more off-targets. Said penalty may be applied to candidate molecules stored in memory 104 and said penalty may be recorded as a penalty term value linked to a candidate molecule identifier. Said penalty may be applied during scoring prior to parameter updating so that parameter updates reflect penalized score values. The penalty recording may allow traceable accounting of constraint violations in memory 104 and may enable later auditing of ranking decisions of candidate molecules based on constraint impact.

In an embodiment, the toxicity constraint penalty may reduce the feedback score when a predicted toxicity risk output exceeds a stored limit and said similarity constraint penalty may reduce the feedback score when a similarity measure relative to molecules in database 106 exceeds a stored similarity limit. Said synthetic feasibility constraint penalty may reduce the feedback score when a synthetic feasibility score falls below a stored feasibility threshold. Said target-specificity constraint penalty may reduce the feedback score when predicted activity for an intended biological target falls below a stored minimum. A selectivity constraint penalty may reduce the feedback score when predicted off-target interaction exceeds a stored off-target limit. The application of penalty may reduce selection of candidate molecules with unacceptable risk indicators, may reduce generation of candidate molecules that mirror known structures beyond a similarity limit, and may guide iterative updates toward candidate pools with improved feasibility and selectivity without altering candidate molecule generation operations stored in memory 104.

In an embodiment, penalty magnitude may be stored as a constant penalty value, a piecewise penalty value, or a scaled penalty value that grows with degree of violation, and penalty aggregation may sum multiple penalties when multiple constraints are violated. Penalty aggregation rules may be prestored in memory 104 and applied consistently across candidate pools. Application of penalty may operate together with weighted aggregation scoring so that penalized scores influence ranking and parameter updating. Such penalty scaling may support smoother ranking transitions rather than abrupt rejection when minor violations occur.

In another embodiment, one or more processors 102 apply at least one structural alert filter selected from reactive functional group filters, toxicophore filters, mutagenicity alert filters, rule-of-five filters, metal chelator filters, unstable moiety filters, strained ring filters, genotoxicity alert filters, and covalent-binding alert filters to each candidate molecule and each subsequent candidate molecule represented in memory 104. Each structural alert filter may evaluate molecular structure information derived from sequential molecular representations stored in memory 104 and may output an alert indicator that is stored in memory 104. A preset number for alert triggering may be stored in memory 104 and used as an exclusion threshold. The alert indicator storage may enable transparent tracking of which filters trigger for a given candidate molecule. Such alert indicator storage may allow later analysis of filter prevalence across candidate pools and may enable filter tuning through stored filter rule updates.

In another embodiment, one or more processors 102 exclude each candidate molecule and each subsequent candidate molecule whenever at least a preset number of structural alert filters are triggered, and one or more processors 102 generate a plurality of excluded molecules stored in memory 104. Exclusion may occur prior to ranking so that excluded molecules do not enter a ranked candidate set, and exclusion may occur prior to parameter updating so that excluded molecules do not influence generative parameter updates. Exclusion thresholding may operate as a single-trigger exclusion or as a multi-trigger exclusion depending on preset number stored in memory 104. Such exclusion behavior reduces processing and update influence from candidate molecules associated with safety-related alerts or unstable motifs and promotes convergence toward candidate pools with fewer structural alerts.

In another embodiment, one or more processors 102 compute non-excluded feedback scores for each candidate molecule and each subsequent candidate molecule that is not in the plurality of excluded molecules. Non-excluded feedback score computation may use the same drug-property evaluation model, score rules stored in memory 104 while omitting excluded molecules from score aggregation and ranking. Non-excluded feedback scores may be stored in memory 104 as a non-excluded score list linked to candidate molecule identifiers and filter-trigger counts. The non-excluded scoring enables ranking and parameter updating based on candidate molecules that satisfy alert thresholds and permits stable batch statistics and selection rules to operate on candidate molecules with reduced alert burden.

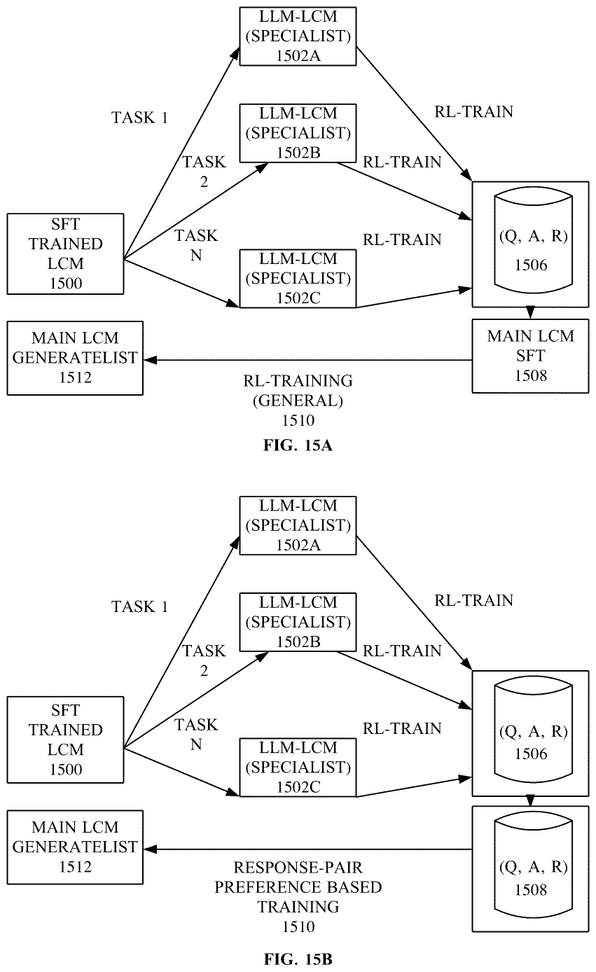

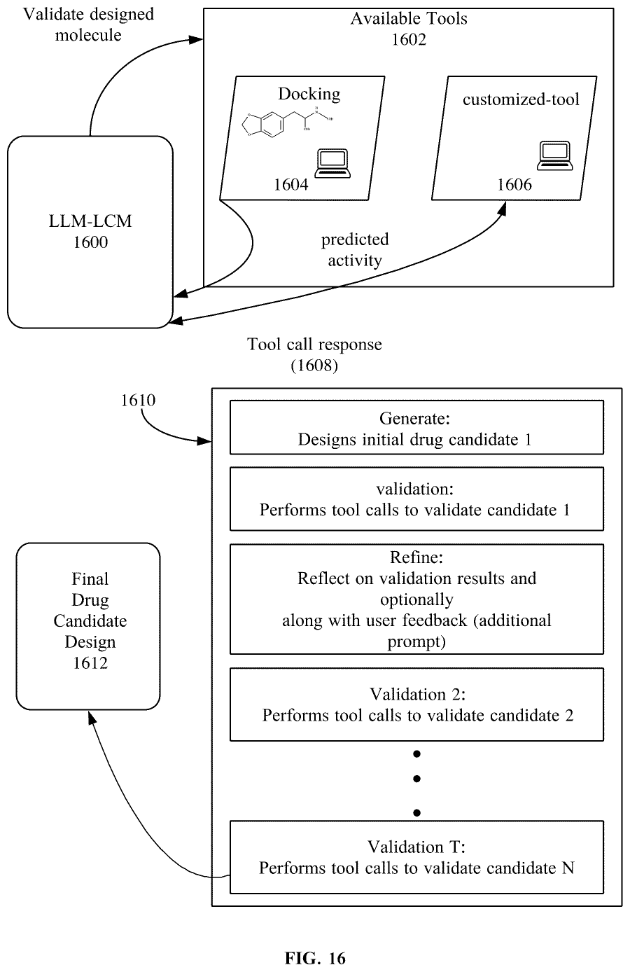

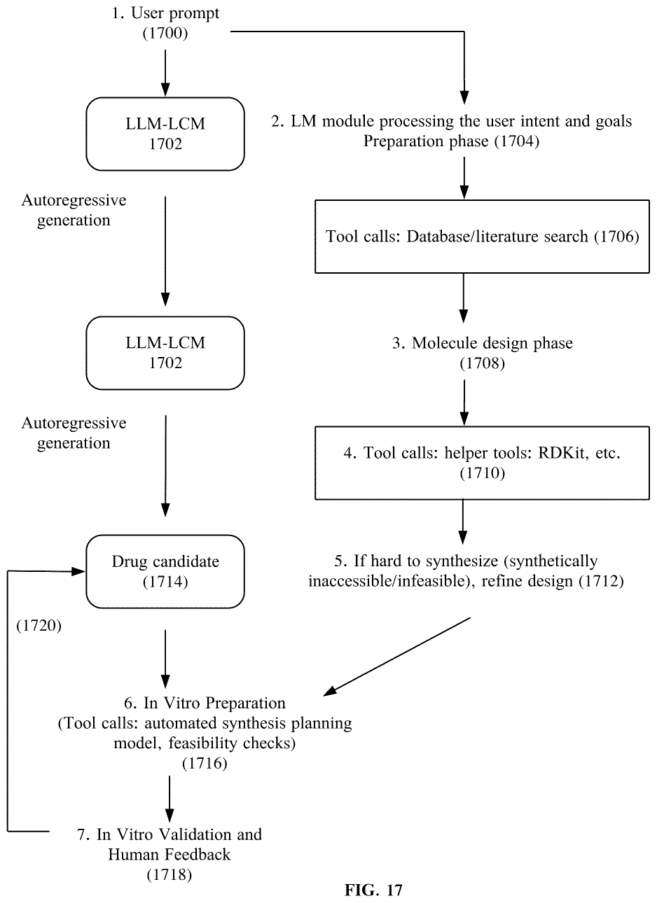

In some embodiments, system 100 implements an integrated LLM-LCM architecture wherein processor 102 executes both a large language model (LLM) component and a large chemistry model (LCM) component that cooperatively process natural language instructions and molecular data. Said integrated LLM-LCM architecture may comprise a shared transformer model with separate encoders for representing molecules in a molecular embedding space and natural language instructions in a language embedding space, wherein cross-attention mechanisms process relationships between the two modalities. In an aspect, processor 102 receives a natural language user prompt specifying drug design objectives, target biological properties, or constraints on candidate molecule characteristics, and processor 102 interprets said user prompt using the LLM component to configure parameters of the generative chemistry language model for subsequent candidate molecule generation. In some cases, processor 102 invokes external tools through an agentic tool-calling interface, said external tools comprising molecular docking engines, database query tools, literature search tools, or synthesis planning tools as described with reference to FIG. 16. Processor 102 receives tool call responses from said external tools and incorporates information from said tool call responses into subsequent candidate molecule generation or refinement operations. In an embodiment, system 100 supports a multi-step automated drug discovery workflow as described with reference to FIG. 17, wherein processor 102 iteratively performs preparation phases, molecule design phases, and validation phases until candidate molecules satisfying predetermined criteria are identified. Said multi-step workflow may further comprise in-vitro preparation stages wherein processor 102 generates synthesis plans for promising candidate molecules and in-vitro validation stages wherein processor 102 receives experimental assay results and updates model parameters based on said experimental results. In some aspects, processor 102 trains multiple target-specific specialist generative chemistry language models as described with reference to FIGS. 15A and 15B, each specialist model optimized for a respective biological target, and processor 102 performs self-distillation to combine knowledge from said specialist models into a generalist model capable of generating candidate molecules across multiple drug targets. Memory 104 stores model parameters, training data, candidate molecule records, validation results, and workflow state information supporting said integrated processing operations performed by system 100.

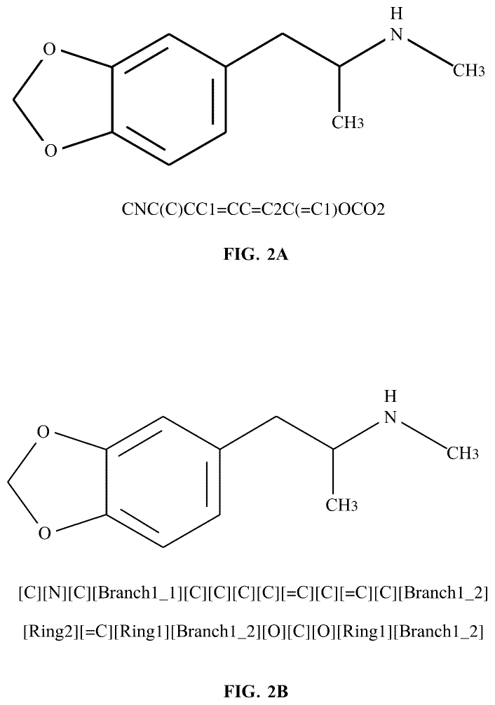

FIGS. 2A and 2B illustrate a molecular representation system demonstrating conversion of molecular structure information of a training molecule into a sequential molecular representation, in accordance with embodiments of the present disclosure. FIG. 2A shows a structural representation of the training molecule depicting atoms, bonds, ring systems, and branching relationships derived from molecular structure information stored in memory 104, and FIG. 2B shows a corresponding linearized character sequence generated from said molecular structure information as said sequential molecular representation selected from a simplified molecular input line entry system (SMILES) string and a self-referencing embedded string (SELFIES), wherein the linearized character sequence represents atoms, bonds, ring closures, branching indicators, and stereochemical indicators arranged under a selected encoding grammar, thereby illustrating how processor 102 converts normalized and canonically ordered molecular structure information into a uniform string form suitable for tokenization, association with a corresponding training molecule identifier, and use as model input during training and scoring operations.

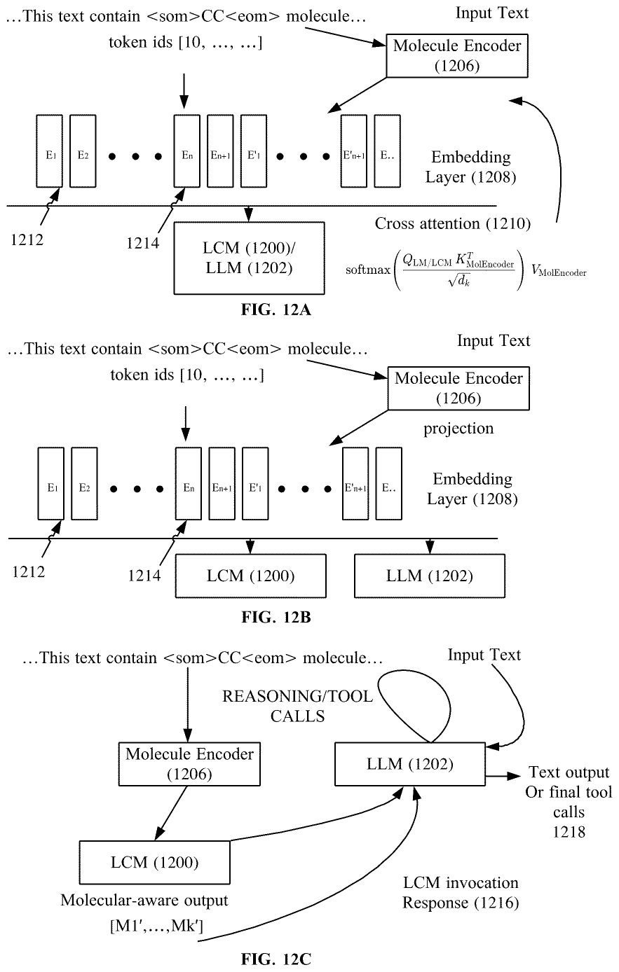

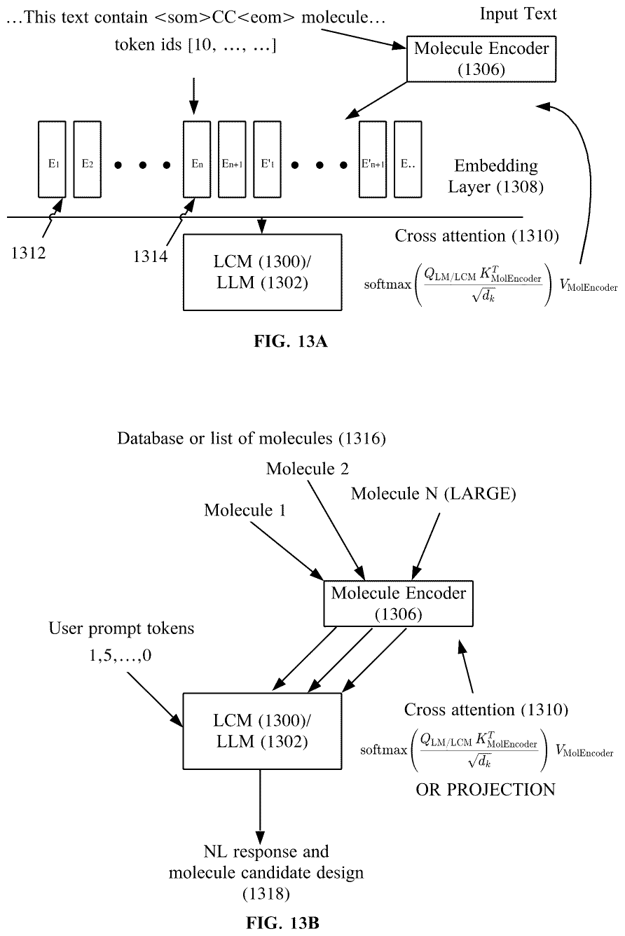

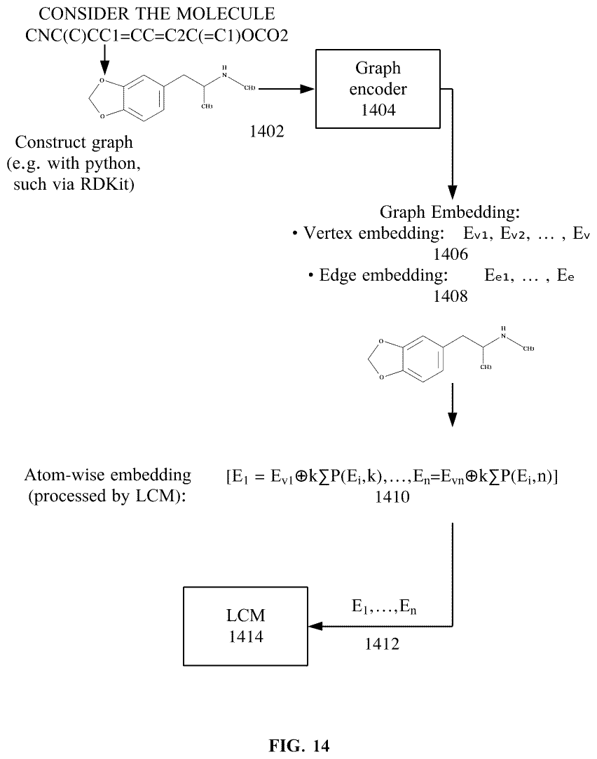

In some embodiments, the molecular representation system illustrated in FIGS. 2A and 2B operates in conjunction with a molecular encoder component as described with reference to FIGS. 12A, 12B, 12C, 13A, 13B, and 14 to generate molecular embeddings that augment the sequential molecular representations. Processor 102 processes each sequential molecular representation through a molecular encoder 1206 that produces embedding vectors capturing chemical and structural features not explicitly encoded in the SMILES or SELFIES token sequence. Said embedding vectors may encode three-dimensional stereochemical information, relative spatial positions of atoms, or learned chemical property representations derived from pretraining on large molecular databases. In an aspect, processor 102 performs token-wise injection wherein each token of the sequential molecular representation is associated with a corresponding molecular embedding vector, said molecular embedding vector added to or concatenated with the token embedding prior to processing by transformer layers of the generative chemistry language model or drug-property evaluation model. In some cases, processor 102 performs sequence-wise injection wherein the molecular encoder produces a contextualized embedding vector for the entire molecular sequence, said contextualized embedding vector injected into the embedding space of a large language model component to enable joint processing of molecular and natural language information as described with reference to FIGS. 13A and 13B. In an embodiment, processor 102 constructs a molecular graph representation from the sequential molecular representation, said molecular graph representation comprising vertices corresponding to atoms and edges corresponding to bonds, and processor 102 processes said molecular graph representation through a graph encoder 1404 as described with reference to FIG. 14 to produce atom-wise embeddings 1410 that are subsequently mapped to the sequential token positions. Said graph-based encoding may capture topological relationships and molecular connectivity patterns that complement the sequential information encoded in SMILES or SELFIES representations. Processor 102 stores the sequential molecular representations, associated molecular embeddings, and graph-derived embeddings in memory 104 as linked data structures supporting flexible retrieval during training, evaluation, and generation operations performed by system 100.

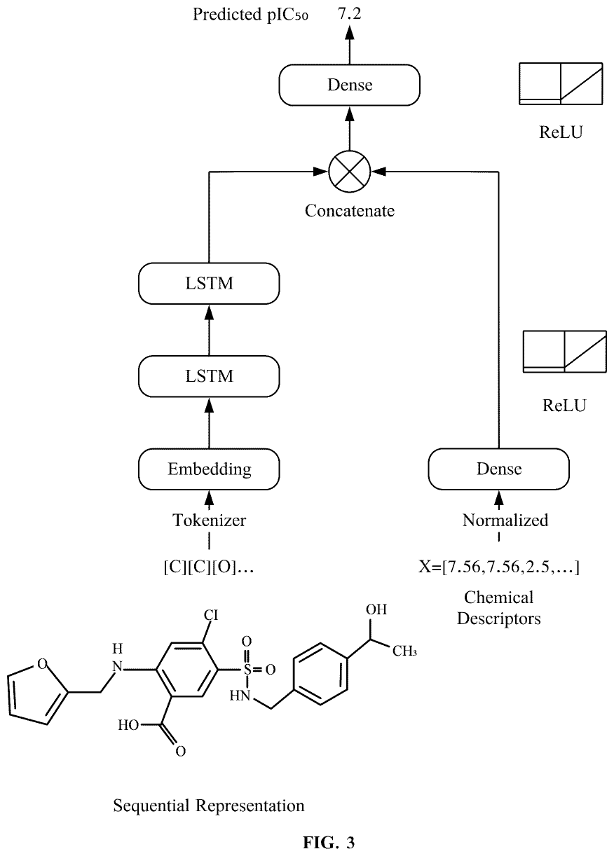

FIG. 3 illustrates an exemplary diagram for processing performed by a drug-property evaluation model that receives a sequential molecular representation and a numerical chemical descriptor and produces an output indicative of at least one predicted drug property, in accordance with embodiments of the present disclosure. In FIG. 3, said sequential molecular representation stored in memory 104, said sequential molecular representation comprising a SMILES string or a SELFIES, is provided as a sequence input. Processor 102 performs tokenization on said sequential molecular representation to generate a token sequence, and processor 102 performs embedding of said token sequence to generate an embedded sequence representation. Processor 102 processes said embedded sequence representation through a plurality of sequence-processing layers represented as LSTM layers to generate a sequence feature representation associated with said sequential molecular representation. In parallel, said numerical chemical descriptor stored in memory 104 is provided as a descriptor input, where said numerical chemical descriptor comprises at least one of a physicochemical descriptor, a structural descriptor, a topological descriptor, a molecular fingerprint, a fragment descriptor, or a learned descriptor embedding. Processor 102 performs normalization on said numerical chemical descriptor to generate a normalized descriptor vector, and processor 102 processes said normalized descriptor vector through a dense layer and a ReLU stage to generate a descriptor feature representation. Processor 102 performs concatenation that joins said sequence feature representation and said descriptor feature representation to generate a fused feature representation. Processor 102 processes said fused feature representation through a dense layer to generate an output value indicative of at least one predicted drug property, where FIG. 3 shows a predicted pIC₅₀ output as an example, and where said output value enables derivation of a feedback score used for ranking of candidate molecules and for iterative updating of a generative chemistry language model.

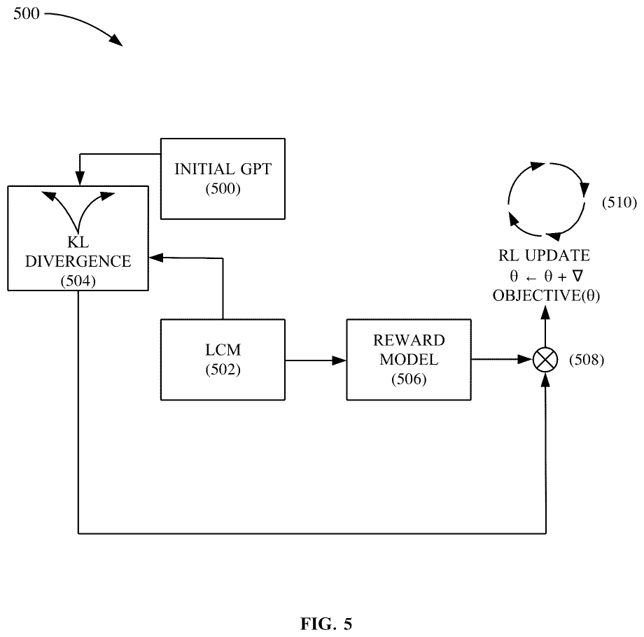

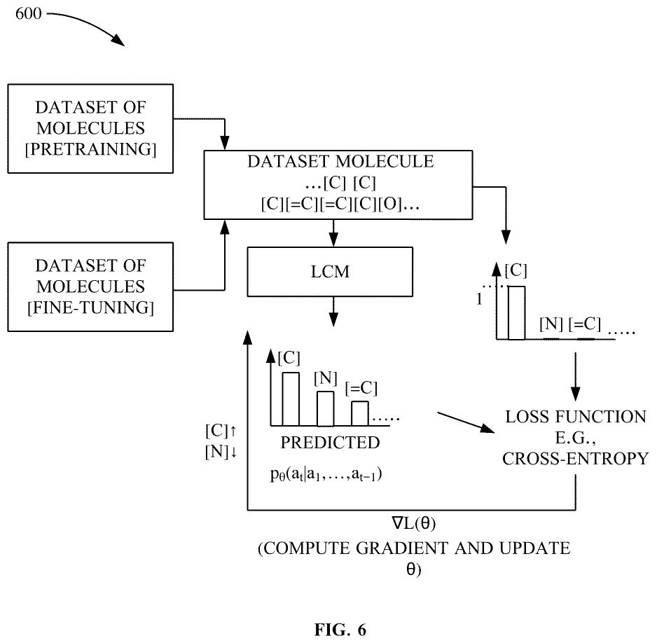

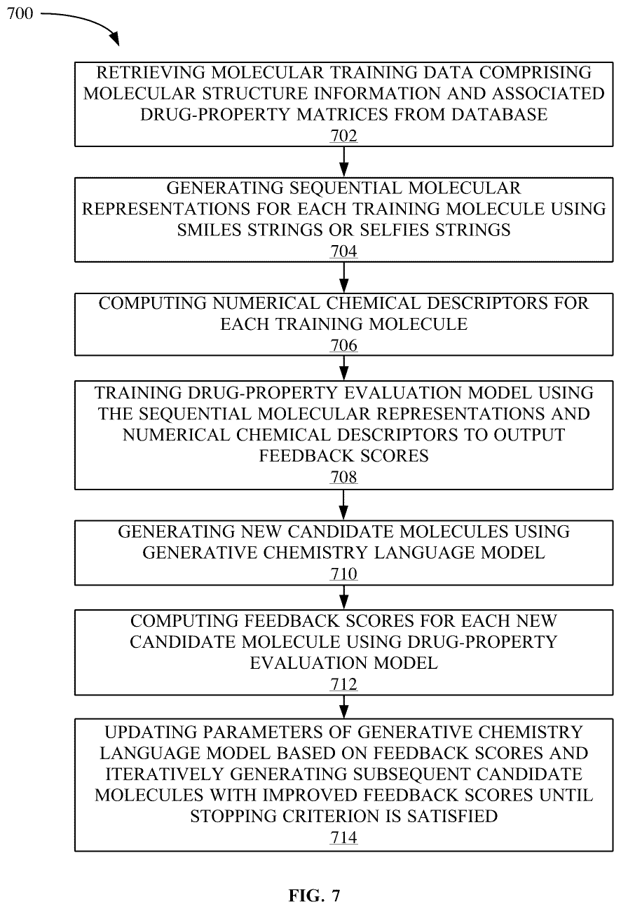

In some embodiments, the drug-property evaluation model illustrated in FIG. 3 operates as a reward model within the reinforcement learning framework described with reference to FIGS. 5 and 6, wherein the predicted drug property outputs are transformed into feedback scores that guide parameter updates of the generative chemistry language model. Processor 102 computes feedback scores by applying reward transformation functions to the predicted drug property outputs, said reward transformation functions comprising linear scaling, logarithmic transformation, or threshold-based step functions that map predicted property values to reward magnitudes suitable for policy optimization. In an aspect, the drug-property evaluation model comprises a quantitative structure-property relationship (QSPR) predictor that is trained on experimental activity labels associated with specific biological targets, enabling target-specific feedback score computation for candidate molecules generated during the molecule design phase 708 of FIG. 7. In some cases, processor 102 trains multiple drug-property evaluation models, each drug-property evaluation model trained to predict a different drug property or trained on activity data for a different biological target, and processor 102 computes a multi-objective feedback score by aggregating outputs from said multiple drug-property evaluation models using weighted combination, Pareto ranking, or constraint satisfaction methods. Said multi-objective feedback score may balance potency components reflecting predicted binding affinity or inhibitory activity with developability components reflecting predicted ADMET properties, synthesizability, or novelty relative to known compounds. In an embodiment, the drug-property evaluation model is periodically updated during the iterative training process based on experimental assay results obtained from synthesized candidate molecules as described with reference to FIG. 17, thereby incorporating empirical validation data into subsequent feedback score computations. Processor 102 stores trained drug-property evaluation model parameters, reward transformation function configurations, and multi-objective aggregation weights in memory 104, supporting flexible configuration of feedback score computation across different drug design objectives and biological targets. In some aspects, the drug-property evaluation model further receives validation results from external in-silico validation tools as described with reference to FIG. 16, and processor 102 modifies feedback scores based on said validation results to produce modified feedback scores that reflect both predicted properties and external validation outcomes.

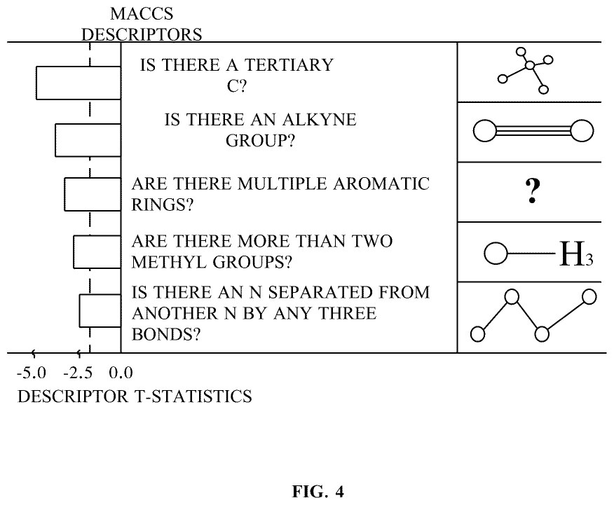

FIG. 4 illustrates an exemplary diagram for interpretation of a molecular fingerprint descriptor used as a numerical chemical descriptor, in accordance with embodiments of the present disclosure. FIG. 4 depicts a plurality of fingerprint descriptor elements expressed as structural-query conditions, where each fingerprint descriptor element corresponds to presence or absence of a structural pattern in a molecule. FIG. 4 further depicts a descriptor significance measure for said fingerprint descriptor elements, where a magnitude of a descriptor statistic indicates an extent of association between a fingerprint descriptor element and an output quantity used during training or evaluation of a drug-property evaluation model. FIG. 4 further depicts representative structural icons corresponding to example fingerprint descriptor elements, where such icons illustrate example patterns such as a tertiary carbon pattern, an alkyne pattern, multiple aromatic ring patterns, a methyl group count pattern, or a nitrogen separation pattern. In operation, processor 102 stores such fingerprint descriptor elements as fixed-length bit vectors or fixed-length count vectors in memory 104, and processor 102 uses such fingerprint descriptor elements as part of the plurality of numerical chemical descriptors for training and scoring. FIG. 4 enables selection, weighting, or filtering of fingerprint descriptor elements for reduced redundancy, improved score stability, and improved interpretability of descriptor contributions during evaluation of candidate molecules.

In some embodiments, the molecular fingerprint descriptors illustrated in FIG. 4 are computed in conjunction with the sequential molecular representations described with reference to FIGS. 2A and 2B to form combined input representations for the drug-property evaluation model of FIG. 3. Processor 102 computes molecular fingerprint descriptors for each training molecule retrieved from database 106 and for each candidate molecule generated by the generative chemistry language model, storing said fingerprint descriptors in memory 104 as numerical chemical descriptor records linked to corresponding molecular structure information. In an aspect, the molecular fingerprint descriptors support computation of similarity scores between generated candidate molecules and reference molecules within database 106, said similarity scores incorporated into feedback score computation to encourage novelty relative to known compounds or to penalize excessive similarity to compounds associated with toxicity alerts or intellectual property constraints. In some cases, processor 102 utilizes molecular fingerprint descriptors during the validation phase described with reference to FIG. 16, wherein fingerprint-based similarity searches against chemical databases are performed through database query tool calls invoked by the integrated LLM-LCM system 1600. Said database query tool calls may retrieve structurally similar compounds with known experimental activity data, enabling the LLM-LCM system to reason about predicted properties of generated candidate molecules based on activity profiles of similar known compounds. In an embodiment, processor 102 computes fingerprint-based diversity metrics across the plurality of candidate molecules generated during each iteration of the training process described with reference to FIGS. 5 and 6, said diversity metrics supporting assessment of whether the generative chemistry language model explores diverse regions of chemical space or converges toward a narrow structural family. Processor 102 may incorporate said diversity metrics into stopping criterion evaluation, wherein satisfaction of a diversity condition across a subset of candidate molecules triggers termination of the iterative parameter updating process. In some aspects, the molecular fingerprint descriptors are further utilized during the preparation phase 704 of the multi-step drug discovery workflow illustrated in FIG. 17, wherein processor 102 performs fingerprint-based searches to identify known compounds targeting the same biological target specified in the natural language user prompt 1700, said known compounds providing reference structures that inform subsequent candidate molecule generation during the molecule design phase 708.

FIG. 5 illustrates a policy optimization and divergence-controlled updating framework (500) for a generative chemistry language model (502), in accordance with embodiments of the present disclosure. FIG. 5 shows an initial generative parameter set corresponding to an initial GPT model (500), a ChemGPT model (502) executed by processor 102 to generate candidate molecule sequences, a reward model (506) configured to generate feedback scores for said candidate molecule sequences, and a policy optimization stage (508) configured to perform reinforcement learning based updating of one or more parameters using feedback scores stored in memory 104, wherein parameter updating is represented by θ←θ+∇ Objective(θ) (510). FIG. 5 further shows application of Kullback-Leibler regularization (504) relative to a reference policy to compute divergence between an updated policy and a baseline policy and to constrain parameter drift during iterative updating. FIG. 5 further illustrates integration of reward-based gradients and divergence-based penalty terms within a combined objective (508) so that generation probability for higher-scoring candidate molecule sequences increases relative to lower-scoring candidate molecule sequences while maintaining distribution stability. The framework depicted in FIG. 5 supports repeated update iterations (510), convergence-controlled stopping, and balanced optimization between feedback score maximization and reference-policy divergence control (504).

In some embodiments, the policy optimization and divergence-controlled updating framework illustrated in FIG. 5 operates in conjunction with the multi-specialist training framework described with reference to FIGS. 15A and 15B to produce both target-specific specialist models and generalist models capable of designing candidate molecules across multiple biological targets. Processor 102 initializes multiple instances of the ChemGPT model 502, each instance trained using the policy optimization stage 508 with a reward model 506 configured for a respective biological target or optimization objective, thereby producing a plurality of LLM-LCM specialists 1502A, 1502B, 1502C as illustrated in FIG. 15A. In an aspect, processor 102 performs self-distillation by aggregating question-answer-reward tuples 1506 generated by said specialist models and training a main LCM generalist 1512 through supervised fine-tuning followed by additional RL-training 1510, said generalist model exhibiting cross-task generalization across different drug targets and optimization objectives. In some cases, the policy optimization algorithm applied during the RL update 510 comprises group relative policy optimization (GRPO), wherein processor 102 samples multiple candidate molecule outputs for each input query and computes advantages relative to the group of sampled outputs rather than requiring a separate value function estimator. Said GRPO-based optimization may reduce computational requirements while maintaining stable policy improvement across training iterations. In an embodiment, processor 102 dynamically adjusts the KL divergence 504 regularization coefficient during training based on observed divergence magnitudes between the current ChemGPT model 502 and the initial GPT model 500, said dynamic adjustment supporting adaptive control of exploration-exploitation balance as the model progresses through different phases of the optimization process. Processor 102 stores policy optimization hyperparameters, KL divergence coefficients, and training checkpoints in memory 104, enabling resumption of training from intermediate states and comparison of model performance across different optimization configurations. In some aspects, the policy optimization framework further incorporates feedback from external in-silico validation tools as described with reference to FIG. 16, wherein processor 102 modifies reward signals based on validation outcomes from molecular docking tools 1604 or customized tools 1606 prior to computing the RL update 510, thereby integrating external validation into the policy optimization loop. Said integration of external validation may guide the generative chemistry language model toward candidate molecules that satisfy both predicted property criteria evaluated by the reward model 506 and structural or interaction criteria evaluated by external computational tools.

FIG. 6 illustrates an exemplary diagram (600) for training of a generative chemistry language model using sequential molecular representations, in accordance with embodiments of the present disclosure. FIG. 6 shows a dataset of molecules for pretraining and a dataset of molecules for fine-tuning. Processor 102 obtains molecule records from said dataset of molecules for pretraining and said dataset of molecules for fine-tuning and forms, for each molecule record, a dataset molecule sequential representation expressed as an ordered token sequence corresponding to a SMILES string or a SELFIES, as depicted by token forms such as “. . . [C][C][C]═[C]═[C][C][O] . . . ”. Said dataset molecule sequential representation is supplied as an input sequence to the generative chemistry language model, where the generative chemistry language model performs causal sequence processing so that, for each token position, a distribution over a next token is produced based on prior tokens in the sequence.

FIG. 6 further depicts predicted token probabilities represented as a probability distribution over candidate next tokens, where said probability distribution is indicated using a conditional probability form p_{θ}(aₜ|a₁, . . . , aₜ₋₁). FIG. 6 further depicts a reference next token associated with a ground-truth continuation of the dataset molecule sequential representation, where the reference next token corresponds to a token present in a training sequence at a next position. During supervised sequence training, processor 102 compares predicted token probabilities against the reference next token for each token position across a sequence. Such comparison occurs across many sequences from the dataset of molecules for pretraining and the dataset of molecules for fine-tuning so that parameter values of the generative chemistry language model are adjusted based on aggregate prediction mismatch.

FIG. 6 further depicts a loss function, such loss function comprising a cross-entropy loss in one arrangement. The loss function receives predicted token probabilities and the reference next token and outputs a loss value that quantifies mismatch between predicted token probabilities and the reference next token. Processor 102 accumulates loss values across token positions of a sequence and across a batch of sequences to form a batch loss value. FIG. 6 further depicts computation of a gradient of the loss with respect to parameter values θ and updating of parameter values θ using the computed gradient so that, across training iterations, probability mass assigned to a reference next token increase. Pretraining supports learning of token grammar and token semantics for sequential molecular representations across a broad molecular distribution, and fine-tuning supports adaptation of the generative chemistry language model toward sequential molecular representations associated with drug-like molecules represented in the fine-tuning dataset.

In some embodiments, the training methodology illustrated in FIG. 6 operates as part of a multi-stage training pipeline that progresses from supervised fine-tuning through reinforcement learning optimization as described with reference to FIGS. 5, 15A, and 15B. Processor 102 initially trains the generative chemistry language model using supervised fine-tuning (SFT) on molecular sequences retrieved from database 106, said SFT stage establishing baseline molecular generation capabilities prior to reinforcement learning optimization. In an aspect, the training methodology of FIG. 6 incorporates curriculum learning wherein processor 102 progressively increases task difficulty during training by adjusting target property thresholds, introducing additional optimization objectives, or transitioning from single-property optimization to multi-objective optimization. Said curriculum learning may improve training stability and enable the generative chemistry language model to acquire foundational molecular generation skills before addressing more challenging drug design objectives. In some cases, processor 102 implements the training methodology using batched generation wherein multiple candidate molecules are generated in parallel during each training iteration, and processor 102 computes feedback scores for said batch of candidate molecules using the drug-property evaluation model of FIG. 3 before performing a single parameter update based on aggregated gradient information. Said batched generation may improve computational efficiency by amortizing model inference costs across multiple candidate molecules and may reduce variance in gradient estimates during policy optimization.

In an embodiment, processor 102 monitors training progress by computing and storing training metrics in memory 104, said training metrics comprising mean feedback score per iteration, feedback score variance, percentage of candidate molecules exceeding predetermined property thresholds, and molecular validity rate as assessed through SMILES or SELFIES parsing. Processor 102 evaluates said training metrics against stopping criteria, including convergence of feedback scores, reaching of a maximum number of update iterations, or identification of candidate molecules satisfying a plurality of drug-property constraints. In some aspects, the training methodology further supports checkpoint-based model selection wherein processor 102 stores model parameters at predetermined iteration intervals and subsequently selects a final model based on performance evaluation against a held-out validation set of molecules with known experimental activity labels. Said checkpoint-based selection may identify model states that generalize well to unseen molecules while avoiding overfitting to the training distribution. Processor 102 may further apply the training methodology iteratively across multiple rounds as illustrated in the validation loop 1610 of FIG. 16, wherein each round incorporates updated training data derived from external validation results or experimental assay outcomes as described with reference to FIG. 17.

FIG. 7 illustrates a method for designing a drug-like candidate compound, in accordance with the embodiments of the present disclosure. At step 702, a plurality of molecular training data is retrieved from a database, where said plurality of molecular training data comprises molecular structure information for a plurality of training molecules and a plurality of drug-property matrices associated with said plurality of training molecules. Retrieval organizes said molecular structure information and said drug-property matrices into aligned records so that each training molecule has a corresponding set of property values. Retrieval further establishes record identifiers and indexing used for later processing stages, and retrieval may apply selection rules that determine which training molecules and which drug-property matrices are used for subsequent training.

At step 704, for each training molecule in said plurality of training molecules, a sequential molecular representation is generated, where said sequential molecular representation is selected from a plurality of Simplified Molecular Input Line Entry System (SMILES) strings and a plurality of Self-Referencing Embedded Strings (SELFIES), thereby forming a plurality of sequential molecular representations. Generation converts molecular structure information into a character sequence representation that carries atom connectivity and bond information in a linear form. Generation further stores each sequential molecular representation in association with a corresponding training molecule identifier so that later training uses matched representation records.

At step 706, a numerical chemical descriptor associated with each training molecule in said plurality of training molecules is computed to form a plurality of numerical chemical descriptors. In alternative embodiments, the numerical chemical descriptor associated with each training molecule in said plurality of training molecules is collected. Computation derives numeric values or numeric vectors from molecular structure information so that molecular characteristics are expressed in a machine-readable numeric form. Computation produces descriptor vectors aligned to the same training molecule identifiers used for said plurality of sequential molecular representations. Further computation stores said plurality of numerical chemical descriptors so that paired inputs for each training molecule remain available for subsequent model training operations.

At step 708, a drug-property evaluation model is trained using said plurality of sequential molecular representations and said plurality of numerical chemical descriptors, where said drug-property evaluation model is trained to output the feedback score indicative of at least one predicted drug property associated with said plurality of drug-property matrices. Training forms paired samples by linking, for each training molecule, said sequential molecular representation, said numerical chemical descriptor, and one or more property values derived from a drug-property matrix. Training adjusts model parameter values based on error between predicted property outputs and reference property values so that feedback score outputs correlate with drug-property matrices.

At step 710, using a generative chemistry language model, at least one new candidate molecule is generated to form a plurality of new candidate molecules. Generation produces one or more molecular sequences expressed in a sequential format compatible with said plurality of sequential molecular representations, thereby allowing candidate molecules to be processed using the same representation used for training molecules. Generation forms a candidate pool for a given iteration, and generation stores each new candidate molecule as a sequence record with an identifier so that subsequent evaluation and scoring operations are applied consistently across said plurality of new candidate molecules.

At step 712, using said drug-property evaluation model, a feedback score is computed for each new candidate molecule in said plurality of new candidate molecules to form a plurality of feedback scores. Computation processes each new candidate molecule as an input to the drug-property evaluation model and produces at least one predicted drug-property output that is converted into the feedback score. Computation stores each feedback score in association with the corresponding new candidate molecule identifier to support ranking of candidate molecules and to support parameter updating of the generative chemistry language model. Computation may also derive batch statistics from said plurality of feedback scores for monitoring iterative progress.

At step 714, one or more parameters of said generative chemistry language model are updated based on said plurality of feedback scores, where said updating causes said generative chemistry language model to generate the subsequent candidate molecule to form a plurality of subsequent candidate molecules having an improved feedback score relative to said plurality of new candidate molecules. Updating applies score-driven adjustment so that candidate molecule sequences associated with higher feedback scores receive higher generation likelihood relative to lower-scoring sequences. Updating and subsequent candidate generation are iteratively repeated, with feedback score computation performed for each iteration, until at least one stopping criterion is satisfied, where said stopping criterion may correspond to a score threshold, an iteration limit, convergence of feedback scores, satisfaction of drug-property constraints, or a diversity condition across candidate molecules.

In an embodiment, said synthetic feasibility score may be computed for each candidate molecule represented as the sequential molecular representation stored in memory 104, and the feedback score stored in memory 104 may be updated in response to said synthetic feasibility score. Processor 102 may derive said synthetic feasibility score from molecular structure information reconstructed from said sequential molecular representation, and processor 102 may associate said synthetic feasibility score with a candidate molecule identifier stored in memory 104. Said synthetic feasibility score may reflect estimated practicality of preparing said candidate molecule using available starting materials, available reaction patterns, and chemical complexity measures derived from structure. Such synthetic feasibility scoring reduces progression of candidate molecules that may be difficult to prepare and enables alignment between computational ranking and laboratory practicality at an earlier stage of candidate selection.

In an embodiment, computation of said synthetic feasibility score may use one or more rule sets stored in memory 104 that evaluate structural complexity, ring strain indicators, stereochemical burden, reactive group burden, and step-count estimates. Processor 102 may compute a fragment-based score using fragment occurrence frequency derived from training molecules stored in database 106, and processor 102 may compute a route-likelihood score using reaction transform rules prestored in memory 104. Processor 102 may also compute an availability score using building-block presence flags obtained through a database query executed against database 106. Such multi-factor scoring may allow differentiation between candidate molecules with similar predicted potency yet different preparation difficulty.

In an embodiment, updating said feedback score in response to said synthetic feasibility score may apply penalty scaling when said synthetic feasibility score falls below a feasibility threshold stored in memory 104, and updating of said feedback score may apply reward scaling when said synthetic feasibility score exceeds said feasibility threshold. Processor 102 may store both an unadjusted feedback score and an adjusted feedback score in memory 104 so that ranking behavior may be compared under different weighting selections. Processor 102 may apply said adjusted feedback score during parameter updating for the generative chemistry language model so that subsequent candidate molecules may trend toward higher feasibility patterns.

In another embodiment, upon satisfaction of the stopping criterion stored in memory 104, at least one subsequent candidate molecule may be selected for synthesis with an associated synthetic feasibility score that exceeds said synthetic feasibility score threshold. Processor 102 may identify a subset of candidate molecules stored in memory 104 that satisfy said stopping criterion and may evaluate said subset against said synthetic feasibility score threshold stored in memory 104. Selection may use ranked ordering stored in memory 104 so that the candidate molecule with higher adjusted feedback score satisfying thresholds may receive higher selection priority. Such threshold-based selection reduces selection of candidate molecules that may be impractical for laboratory preparation, reduces downstream iteration caused by synthesis failure due to feasibility barriers, and further improves predictability of laboratory workload by constraining candidate molecules to feasible sets under stored thresholds and stored ranking rules.

In another embodiment, selection for synthesis may operate using multi-stage filtering stored in memory 104, where a first stage may apply structural alert exclusion rules and a second stage may apply synthetic feasibility thresholding and a third stage may apply final ranking based on adjusted feedback scores. Processor 102 may store a selected candidate molecule list in memory 104 together with supporting score components, feasibility score components, and constraint pass indicators so that laboratory handoff records remain traceable. Selection may also apply a diversity condition stored in memory 104 so that selected candidate molecules are not clustered around a single scaffold when multiple scaffolds satisfy scoring and feasibility thresholds. Such multi-stage selection may reduce risk associated with selecting chemically similar candidate molecules, which share the same failure mode.

In another embodiment, synthesis selection may trigger generation of a synthesis packet stored in memory 104 containing the sequential molecular representation, a reconstructed structure record, a feasibility score breakdown, and a ranked position record. Processor 102 may write said synthesis packet to a storage region associated with database 106 so that subsequent assay result records may be linked back to the selected candidate molecule identifier. Processor 102 may also store a synthesis threshold report describing which candidate molecules failed thresholding and which candidate molecules passed thresholding under the stored feasibility threshold. Such packet generation may provide traceable connection between computational selection and laboratory execution and may reduce manual transcription errors by preserving molecular structure information and associated identifiers.

In an embodiment, one or more processors 102 may update at least one of the drug-property evaluation model or the generative chemistry language model based on an experimental assay result of a synthesized subsequent candidate molecule. Database 106 may store said experimental assay result as a property label aligned to candidate molecule identifier, and memory 104 may store a linkage record between said candidate molecule identifier and a generation iteration identifier. Processor 102 may ingest said experimental assay result, compare said experimental assay result to predicted property outputs stored in memory 104, and generate an update dataset record stored in memory 104. Such assay-based updating may reduce mismatch between predicted scores and measured outcomes across subsequent training cycles and may allow recalibration of scoring behavior under real measurement distributions.

In an embodiment, updating of the drug-property evaluation model may use incremental training where newly obtained assay labels stored in database 106 are appended to the training dataset stored in memory 104, and processor 102 may perform fine-tuning using said appended dataset. Processor 102 may apply weighting rules stored in memory 104 so that newly measured assay labels receive controlled influence relative to older labels, thereby limiting abrupt score shifts across iterations. Processor 102 may also store a validation split in memory 104 that isolates new candidate molecules to monitor generalization behavior after updating. The incremental updating may allow steady improvement of evaluation behavior without large parameter drift and may provide preservation of learned structure-property relationships from larger historical datasets stored in database 106.

In an embodiment, updating of the generative chemistry language model may use experimental assay outcomes to modify reward values stored in memory 104, where candidate molecules associated with stronger assay outcomes may be assigned higher reward values and candidate molecules associated with weaker assay outcomes may be assigned lower reward values. Processor 102 may then perform parameter updating based on said reward values so that subsequent candidate molecules may trend toward patterns associated with improved assay outcomes. Processor 102 may store a before-update and after-update candidate distribution summary in memory 104 so that distribution changes can be monitored. Such assay-driven updating may support closing of a loop between laboratory outcomes and generation behavior and may reduce exploration of chemical regions associated with poor experimental response.

In another embodiment, a non-transitory computer-readable storage medium may store program code executable by a hardware processor and said program code when executed by processor 102 may cause processor 102 to execute a computer-implemented method for designing a drug-like candidate compound. Said storage medium may be implemented as a solid-state storage device, flash storage device, magnetic storage device, optical storage device, or other persistent storage that retains program code independent of power state. Memory 104 may store said program code during execution, and database 106 may store datasets accessed during execution.

In another embodiment, said program code may comprise instructions that cause processor 102 to retrieve molecular training data from database 106, generate sequential molecular representations, compute numerical chemical descriptors, train a drug-property evaluation model, generate candidate molecules using the generative chemistry language model, compute feedback scores using said drug-property evaluation model, and update one or more parameters of said generative chemistry language model based on feedback scores until satisfaction of a stopping criterion stored in memory 104. Said program code may also write intermediate results to memory 104 so that scoring and ranking outputs are preserved for later review.

In another embodiment, said program code may also comprise instructions that cause processor 102 to apply threshold-based filters, apply penalty rules, compute feasibility scores, store ranked candidate lists, and store selection packets for synthesis, with such penalty rules stored in memory 104 and such datasets retrieved from database 106. Said program code may further cause processor 102 to ingest experimental assay results and perform model updating based on said experimental assay results, with updated parameters stored in memory 104 and associated records written into database 106.

In some embodiments, the method 700 illustrated in FIG. 7 operates in conjunction with the multi-step automated drug discovery workflow described with reference to FIG. 17, wherein the steps 702-712 correspond to computational processing phases that precede in-vitro preparation 716 and in-vitro validation 718 stages. Processor 102 receives a natural language user prompt 1700 specifying drug design objectives, target biological properties, or structural constraints, and processor 102 interprets said user prompt using the integrated LLM-LCM system 1702 to configure parameters for execution of method 700. In an aspect, the preparation phase 704 of FIG. 17 corresponds to steps 702 and 704 of method 700, wherein processor 102 retrieves molecular training data from database 106 and generates sequential molecular representations and numerical chemical descriptors for training the drug-property evaluation model. In some cases, processor 102 performs database or literature search 706 during the preparation phase to gather relevant background knowledge about the specified biological target, said background knowledge comprising known active compounds, binding site characteristics, or reported structure-activity relationships that inform subsequent candidate molecule generation. Said background knowledge may be incorporated into the training data or used to configure reward model parameters for the policy optimization stage.

In an embodiment, the molecule design phase 708 of FIG. 17 corresponds to steps 708 and 710 of method 700, wherein processor 102 generates candidate molecules using the generative chemistry language model and computes feedback scores using the drug-property evaluation model. Processor 102 may invoke helper tool calls 710 during the molecule design phase, said helper tool calls comprising RDKit-based analysis for property computation, structural alert filtering, or synthetic feasibility scoring. In some aspects, the refinement step 712 of method 700 corresponds to iterative parameter updating wherein processor 102 updates parameters of the generative chemistry language model based on feedback scores and generates subsequent candidate molecules having improved predicted properties. Said refinement may continue through multiple iterations until the drug candidate 714 satisfies predetermined stopping criteria, including identification of candidate molecules exceeding feedback score thresholds, satisfaction of multiple drug-property constraints, or meeting of diversity criteria across the candidate molecule population. Processor 102 stores the drug candidate 714 and associated metadata in memory 104, said metadata comprising feedback scores, predicted property values, structural alert assessments, and synthetic feasibility scores supporting selection of candidates for progression to in-vitro preparation 716 and subsequent experimental validation stages. In an embodiment, method 700 further supports closed-loop refinement wherein processor 102 updates at least one of the drug-property evaluation model or the generative chemistry language model based on experimental assay results obtained from synthesized drug candidates, thereby incorporating empirical validation data into subsequent executions of method 700 for continued optimization toward drug candidates suitable for clinical evaluation.

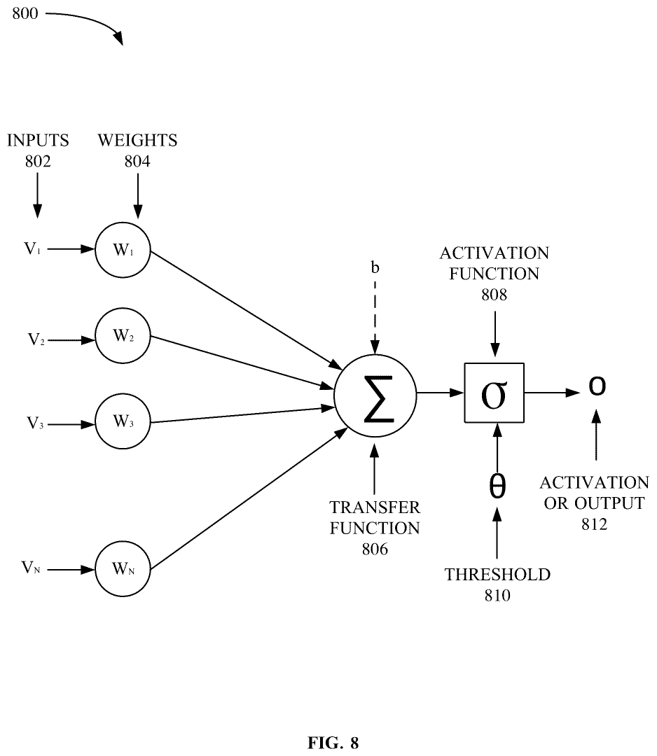

FIG. 8 illustrates exemplary steps for computation of an output value 810 using a single-layer neural processing diagram 800 used within a drug-property evaluation model, in accordance with embodiments of the present disclosure. FIG. 8 shows an input vector 802 and a weight set 804 comprising weight values W1, W2, W3, . . . , WN. In an operation, processor 102 forms input vector 802 using numeric values derived from at least one of the sequential molecular representations stored in memory 104 and a numerical chemical descriptor stored in memory 104. Input vector 802 may contain token-derived embedding values, normalized descriptor values, fused feature values, or a combination thereof, where each element of input vector 802 represents a separate input dimension supplied to diagram 800.

FIG. 8 further illustrates a transfer operation in which each element of input vector 802 is multiplied by a corresponding weight value in weight set 804 to form a plurality of weighted input contributions. Weight magnitudes and weight signs within weight set 804 influence a contribution of each input dimension toward an aggregated net input value. FIG. 8 shows summation stage 806, where summation stage 806 performs aggregation of the weighted input contributions across input vector 802 to generate the net input value. Summation stage 806 corresponds to a dot-product accumulation across input vector 802 and weight set 804.

FIG. 8 further shows a threshold value 812 applied in relation to the net input value generated at summation stage 806. Threshold value 812 is applied as an additive offset to form a shifted net input value or is applied as a comparison reference for the net input value so that a shifted response boundary is established. FIG. 8 shows activation stage 808, where activation stage 808 maps the shifted net input value through a non-linear transformation to generate output value 810. Output value 810 is supplied as an intermediate value for a subsequent neural processing layer or is supplied as a predicted value used for derivation of the feedback score during evaluation of candidate molecules.

In some embodiments, the single-layer neural processing illustrated in FIG. 8 represents a fundamental computational unit that is replicated and interconnected across multiple layers within the drug-property evaluation model described with reference to FIG. 3 and the generative chemistry language model described with reference to FIGS. 5 and 6. Processor 102 executes multiple instances of the single-layer neural processing diagram 800 in sequence, wherein the output value 812 from one layer serves as a component of the input vector 802 for a subsequent layer, enabling hierarchical feature extraction from sequential molecular representations and numerical chemical descriptors. In an aspect, the weight set 804 for each layer is learned during the training process described with reference to FIG. 6, wherein processor 102 adjusts weight values through backpropagation of gradients computed from feedback scores produced by the drug-property evaluation model. Said weight adjustment may employ optimization algorithms comprising stochastic gradient descent, Adam optimization, or AdamW optimization with weight decay regularization. In some cases, the activation stage 808 employs different activation functions for different layers within the neural network architecture, said activation functions comprising rectified linear unit (ReLU) activation for hidden layers, sigmoid activation for output layers producing probability values, or softmax activation for output layers producing probability distributions over discrete token vocabularies as used in the generative chemistry language model. In an embodiment, the threshold value 810 is implemented as a learnable bias parameter that is adjusted during training alongside the weight set 804, said bias parameter enabling the neural processing unit to model affine transformations of the input vector 802. Processor 102 stores the weight set 804, threshold value 810, and activation function configuration for each layer in memory 104 as model parameter records, said parameter records supporting model serialization, checkpoint storage, and transfer learning across different drug design tasks.

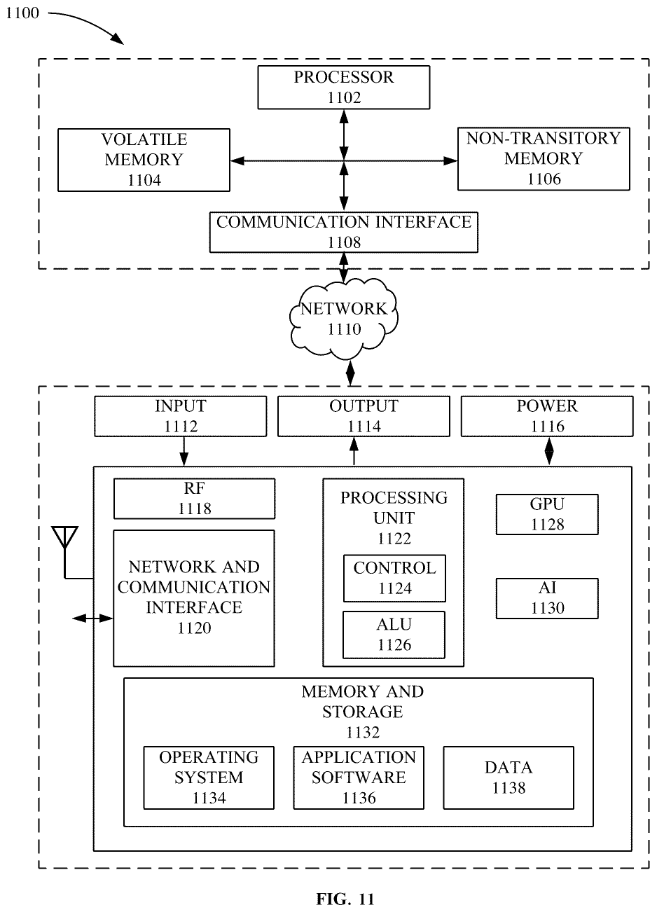

In some aspects, the single-layer neural processing of FIG. 8 is extended to multi-head attention mechanisms within transformer architectures used by the generative chemistry language model, wherein multiple parallel instances of weighted summation and activation are computed across different attention heads, and processor 102 concatenates or aggregates outputs from said attention heads to produce contextualized representations of molecular tokens. Said transformer-based architectures may further incorporate layer normalization, residual connections, and positional encodings that extend the fundamental neural processing operations illustrated in FIG. 8 to support processing of variable-length sequential molecular representations as described with reference to FIGS. 2A and 2B. In an embodiment, the neural processing operations are executed on specialized hardware comprising graphics processing units (GPUs) 1128 or artificial intelligence accelerators 1130 within the computing architecture 1100 described with reference to FIG. 11, said specialized hardware providing parallel computation capabilities that accelerate training and inference operations across large batches of candidate molecules.

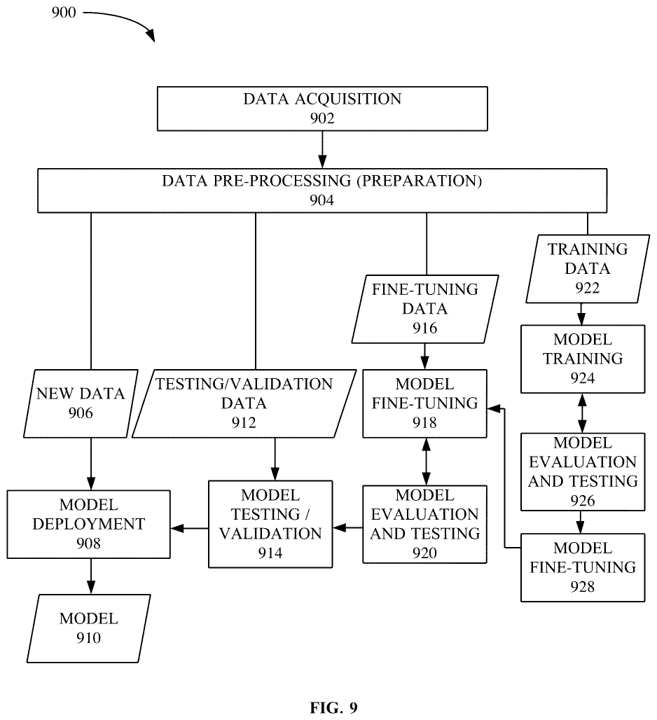

FIG. 9 illustrates exemplary steps for training, fine-tuning, evaluating, validating, testing, selecting, and deploying a machine learning model for a large chemistry model (LCM)-based drug design system, in accordance with embodiments of the present disclosure. The process begins with acquisition of data at a data acquisition stage 902, wherein molecular datasets, property datasets, and associated training resources are obtained, retrieved, generated, or assimilated. The acquired data is subjected to data pre-processing at stage 904, wherein raw data is cleaned, normalized, standardized, transformed, and formatted to produce prepared datasets suitable for downstream processing.

From the data pre-processing stage 904, prepared data may be partitioned into multiple dataset categories, including new data 906, new data 912, training data 922, and fine-tuning data 916. Training data 922 is supplied to a model training stage 924, wherein a machine learning model is trained to learn relationships between molecular representations and corresponding drug-property outcomes. The trained model is evaluated, validated, and tested at a model evaluation and testing stage 926, and results of said evaluation may be fed back to the model training stage 924 for additional training iterations. Upon satisfactory performance, a model fine-tuning stage 928 may be performed to further adapt the trained model using task-specific data.

Fine-tuning data 916 is provided to a model fine-tuning stage 918, wherein parameters of a selected pre-trained model are further adjusted to better suit a specialized task or dataset. Outputs of the model fine-tuning stage 918 are evaluated at a model evaluation and testing stage 920, and results of said evaluation may be iteratively fed back to the model fine-tuning stage 918 to refine parameter values.

Prepared new data 912 may be provided to a model testing and validation stage 914 for assessing generalization performance of the trained or fine-tuned model using data not previously used during training. Prepared new data 906 may be supplied to a model deployment stage 908, wherein a selected trained or fine-tuned model is deployed for operational use. A deployed model 910 is thereby produced and may receive additional new data during runtime, which may be pre-processed using data pre-processing stage 904 prior to inference.

The flow illustrated in FIG. 9 supports iterative improvement of model parameters, controlled selection of optimal model configurations, task-specific adaptation through fine-tuning, unbiased performance evaluation using validation and testing datasets, and deployment of a model capable of generating or evaluating candidate molecules in accordance with stored acceptance thresholds and drug-property objectives.

In some embodiments, the process 900 illustrated in FIG. 9 operates in coordination with the multi-specialist training framework described with reference to FIGS. 15A and 15B to produce both target-specific specialist models and generalist models for drug design. Processor 102 executes process 900 multiple times with different training configurations, each execution producing a target-specific specialist generative chemistry language model, wherein each specialist model is trained using experimental activity labels associated with a respective biological target. In an aspect, the training step 906 of process 900 corresponds to the supervised fine-tuning stage wherein processor 102 trains the model on sequential molecular representations and numerical chemical descriptors as described with reference to FIGS. 2A, 2B, 3, and 4, and the fine-tuning step 912 corresponds to the reinforcement learning optimization stage wherein processor 102 applies policy optimization algorithms as described with reference to FIGS. 5 and 6. Said separation of training and fine-tuning stages enables processor 102 to establish baseline molecular generation capabilities during training step 906 before optimizing for specific drug properties during fine-tuning step 912. In some cases, the evaluation step 908 and validation step 910 of process 900 incorporate external in-silico validation tools as described with reference to FIG. 16, wherein processor 102 invokes molecular docking engines 1604, ADMET predictors, or customized tools 1606 to assess candidate molecules generated by the model under evaluation. Said external validation may provide assessment criteria beyond the predicted properties computed by the drug-property evaluation model of FIG. 3, enabling more comprehensive evaluation of model performance across multiple dimensions relevant to drug discovery.

In an embodiment, the testing step 916 of process 900 assesses model generalization by evaluating performance on held-out test molecules that were not used during training step 906 or fine-tuning step 912, said held-out test molecules comprising molecules with known experimental activity labels retrieved from database 106. Processor 102 computes test metrics comprising prediction accuracy, ranking correlation, and enrichment factors that quantify the model's ability to distinguish active compounds from inactive compounds for the target biological target. In some aspects, the selection step 918 of process 900 implements checkpoint-based model selection wherein processor 102 compares performance metrics across multiple model checkpoints stored during training and selects the checkpoint exhibiting optimal performance on validation data, said selection mitigating overfitting to training data while identifying model states that generalize well to unseen molecules. The deployment step 924 of process 900 corresponds to integration of the trained and validated model into the multi-step automated drug discovery workflow described with reference to FIG. 17, wherein the deployed model operates as the LLM-LCM 1702 that receives natural language user prompts 1700 and generates drug candidates 714 through the molecule design phase 708. In an embodiment, process 900 further supports iterative model refinement wherein processor 102 returns to training step 906 or fine-tuning step 912 upon receiving experimental assay results from synthesized candidate molecules and in-vitro validation step 718 of FIG. 17, said iterative refinement enabling continuous improvement of model performance based on empirical outcome data accumulated across multiple drug discovery campaigns.

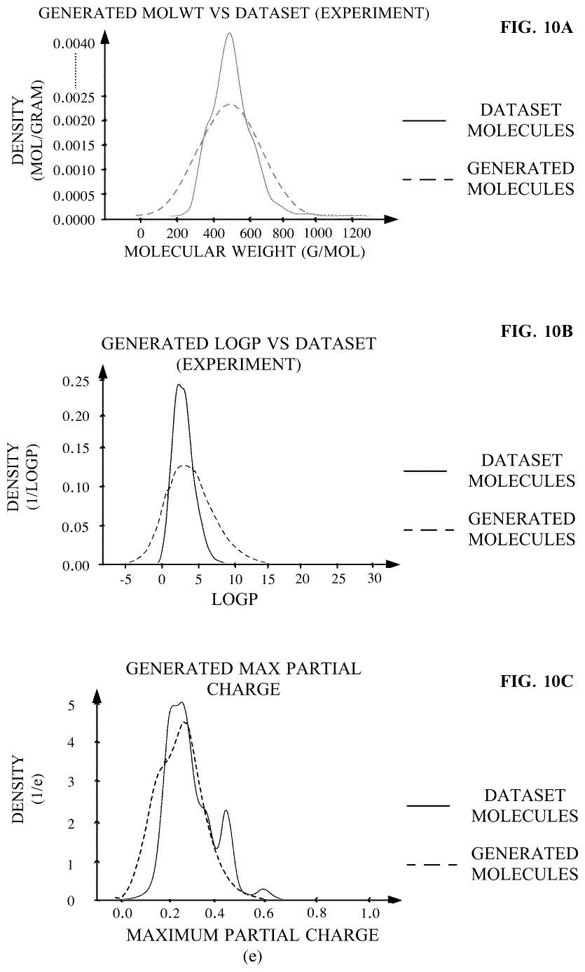

FIGS. 10A, 10B, and 10C illustrate exemplary diagrams for comparison of distributions of numerical chemical descriptors between dataset molecules and generated molecules, in accordance with embodiments of the present disclosure. Three distribution plots denoted as 10A, 10B, and 10C, where each distribution plot presents one or more density curves with density plotted on a vertical axis and a respective molecular property value plotted on a horizontal axis. In FIGS. 10A, 10B, and 10C, a solid line corresponds to dataset molecules derived from molecular structure information stored in database 106, and a dotted line corresponds to generated molecules represented by sequential molecular representations produced by a generative chemistry language model executed by processor 102. Each density curve is normalized to support comparison of distribution shape between dataset molecules and generated molecules without dependence on sample count.

In FIG. 10A, the horizontal axis represents molecular weight expressed in g/mol and the vertical axis represents density. The solid-line density curve corresponds to a molecular weight distribution for dataset molecules, and the dotted-line density curve corresponds to a molecular weight distribution for generated molecules. Overlap between the solid-line density curve and the dotted-line density curve indicates similarity between generated-molecule molecular weight values and dataset-molecule molecular weight values over a dominant molecular weight range. Differences between curve tails represent differences in relative frequency of higher-molecular-weight values or lower-molecular-weight values between dataset molecules and generated molecules under normalization.

In FIG. 10B, the horizontal axis represents log P and the vertical axis represents density. The solid-line density curve corresponds to a log P distribution for dataset molecules, and the dotted-line density curve corresponds to a log P distribution for generated molecules. Overlap between the solid-line density curve and the dotted-line density curve indicates similarity between generated-molecule log P values and dataset-molecule log P values over a dominant log P range. Differences in curve width and tail extent represent differences in relative frequency of lower-log P values or higher-log P values between dataset molecules and generated molecules under normalization.

In FIG. 10C, the horizontal axis represents maximum partial charge expressed in units of e and the vertical axis represents density. The solid-line density curve corresponds to a maximum partial charge distribution for dataset molecules, and the dotted-line density curve corresponds to a maximum partial charge distribution for generated molecules. Overlap between the solid-line density curve and the dotted-line density curve indicates similarity between generated-molecule maximum partial charge values and dataset-molecule maximum partial charge values over a dominant charge range. Differences in curve shape represent differences in relative frequency of higher-charge values or lower-charge values between dataset molecules and generated molecules under normalization.

In an embodiment, processor 102 computes molecular weight values, log P values, and maximum partial charge values as numerical chemical descriptors stored in memory 104 for dataset molecules and generated molecules. In an embodiment, processor 102 generates the density curves using a kernel density estimation procedure executed using descriptor values stored in memory 104 so that discrete descriptor values are represented as continuous density curves for distribution-shape comparison across molecule sets. Comparison of the solid-line density curve to the dotted-line density curve across (A), (B), and (C) supports assessment of whether generated molecules follow descriptor distributions consistent with descriptor distributions present in dataset molecules used during training.

In some embodiments, the distribution comparisons illustrated in FIGS. 10A, 10B, and 10C are computed by processor 102 as part of the evaluation step 908 and validation step 910 of process 900 described with reference to FIG. 9, enabling quantitative assessment of whether generated candidate molecules exhibit property distributions consistent with drug-like chemical space represented in the training data. Processor 102 computes statistical divergence metrics between dataset molecule distributions and generated molecule distributions, said divergence metrics comprising Kullback-Leibler divergence, Jensen-Shannon divergence, or Wasserstein distance, and stores said divergence metrics in memory 104 as model evaluation records. In an aspect, processor 102 monitors distribution alignment across training iterations described with reference to FIG. 6, wherein progressive improvement in distribution alignment indicates that the generative chemistry language model is learning to generate candidate molecules having property profiles similar to known drug-like compounds while the reinforcement learning optimization of FIGS. 5 and 6 guides generation toward regions of chemical space with improved predicted activity. In some cases, processor 102 computes property distributions for numerical chemical descriptors beyond those illustrated in FIGS. 10A, 10B, and 10C, said additional descriptors comprising topological polar surface area, hydrogen bond donor count, hydrogen bond acceptor count, rotatable bond count, and aromatic ring count as described with reference to FIG. 4. Said additional property distributions support comprehensive assessment of candidate molecule compliance with drug-likeness rules including Lipinski Rule-of-Five filters.

In an embodiment, processor 102 generates distribution comparison visualizations and stores said visualizations in memory 104 for presentation to users through output interfaces of the computing architecture 1100 described with reference to FIG. 11, enabling human review of model behavior and identification of property dimensions where generated molecules deviate from desired distributions. Said visualizations may support the human feedback component of the in-vitro validation and human feedback step 718 described with reference to FIG. 17, wherein chemists review generated candidate molecule properties and provide feedback that informs subsequent model refinement. In some aspects, processor 102 utilizes distribution comparison results to implement diversity-aware stopping criteria, wherein processor 102 evaluates whether the plurality of generated candidate molecules satisfies a diversity criterion by assessing coverage of the target property distribution rather than convergence toward a single region of chemical space. Said diversity-aware evaluation may prevent mode collapse wherein the generative chemistry language model generates structurally similar candidate molecules that cluster in a narrow region of chemical space despite having high individual feedback scores. In an embodiment, processor 102 further computes conditional property distributions for candidate molecules stratified by predicted activity level, enabling assessment of whether high-scoring candidate molecules generated during the molecule design phase 708 of FIG. 7 and FIG. 17 exhibit property distributions that differ systematically from the overall generated molecule population or from the training dataset molecules, said conditional analysis supporting identification of property profiles associated with predicted drug efficacy for the target biological target.

FIG. 11 illustrates an exemplary computing architecture for execution of a system to design a drug-like candidate compound, in accordance with embodiments of the present disclosure. In the illustrated architecture, processor 1102 is coupled with volatile memory 1104 and non-transitory memory 1106. Volatile memory 1104 maintains working values generated during execution, such working values comprising token sequences derived from sequential molecular representations, embedding vectors, numerical chemical descriptor vectors, probability distributions, loss values, gradient values, and feedback scores associated with batches of molecules processed during training and evaluation. Non-transitory memory 1106 maintains persistent program instructions and persistent data, such persistent data comprising stored parameter values associated with a generative chemistry language model and a drug-property evaluation model and stored molecular training data comprising molecular structure information and drug-property matrices.

In the illustrated architecture, communication interface 1108 is coupled with processor 1102 and coupled with network 1110. Communication interface 1108 supports transfer of molecular structure information, drug-property matrices, and generated candidate molecule records between processor 1102 and one or more external resources reachable through network 1110. Network 1110 supports access to remote data stores that maintain molecular structure information and drug-property matrices and supports access to external in-silico validation tools that output evaluation results for candidate molecules. Communication interface 1108 receives such evaluation results through network 1110 and stores such evaluation results in volatile memory 1104 or non-transitory memory 1106 for use during feedback score modification and parameter updating operations.

In the illustrated architecture, input (e.g., touch screen) 1112 and output (e.g., display) 1114 are coupled with processing unit 1122 and coupled with power 1116. Input (e.g., touch screen) 1112 receives user inputs associated with candidate molecule generation and selection, such user inputs comprising target identifiers, constraint values, threshold values, diversity criteria, and stopping criterion values stored for use during iterative updating. Output (e.g., display) 1114 presents generated candidate molecules, ranked candidate molecules, predicted drug property values, and feedback score values produced during evaluation operations. Power 1116 supplies operating power to processing unit 1122, GPU 1128, AI 1130, memory and storage 1132, and peripheral interface circuitry.

In the illustrated architecture, RF 1118 and network and communication interface 1120 are coupled with processing unit 1122 and support wireless or wired exchange through network 1110. Processing unit 1122 comprises control 1124 and ALU 1126. Control 1124 manages sequencing of instruction execution for retrieval operations, generation operations, training operations, scoring operations, and parameter updating operations. ALU 1126 performs arithmetic and logical operations for descriptor computation, normalization, feature aggregation, score computation, loss computation, and gradient computation. GPU 1128 performs parallel numeric processing for batch operations applied to sequential molecular representations and numerical chemical descriptors. AI 1130 performs acceleration of neural processing operations used during execution of the drug-property evaluation model and the generative chemistry language model.

In the illustrated architecture, memory and storage 1132 stores operating system 1134, application software 1136, and data 1138. Operating system 1134 supports low-level management of computing resources and peripheral components. Application software 1136 stores program instructions that, when executed by processing unit 1122, perform retrieval of molecular training data, generation of sequential molecular representations, computation of numerical chemical descriptors, training of the drug-property evaluation model, generation of candidate molecules using the generative chemistry language model, computation of feedback scores, and updating of one or more parameters of the generative chemistry language model until satisfaction of at least one stopping criterion. Data 1138 stores molecular structure information, drug-property matrices, sequential molecular representations, numerical chemical descriptors, candidate molecule records, feedback score records, and model parameter records used during operation.

In some embodiments, the computing architecture 1100 illustrated in FIG. 11 provides the hardware infrastructure for executing the drug-like candidate compound design system 100 described with reference to FIG. 1, wherein processor 1102 corresponds to processor 102, memory and storage 1132 corresponds to memory 104, and network and communication interface 1120 supports connectivity to database 106 and external validation tools. The GPU 1128 and AI 1130 components of processing unit 1122 provide specialized computational resources for training and inference operations performed by the drug-property evaluation model described with reference to FIG. 3 and the generative chemistry language model described with reference to FIGS. 5 and 6. In an aspect, GPU 1128 executes parallel matrix multiplication and convolution operations that accelerate the single-layer neural processing described with reference to FIG. 8, enabling efficient computation of weighted summation stage 806 and activation stage 808 across large batches of candidate molecules during training iterations. Said parallel computation capabilities may reduce training time for the reinforcement learning optimization process described with reference to FIGS. 5 and 6 by enabling simultaneous evaluation of multiple candidate molecules generated by the generative chemistry language model. In some cases, AI 1130 comprises dedicated tensor processing units or neural processing units optimized for transformer-based architectures used by the integrated LLM-LCM system described with reference to FIGS. 12A, 12B, 12C, 13A, 13B, and 14, said dedicated hardware providing accelerated attention computation and embedding projection operations that support real-time candidate molecule generation during the molecule design phase 708 of FIG. 7 and FIG. 17. In an embodiment, network 1110 and communication interface 1108 support invocation of external in-silico validation tools as described with reference to FIG. 16, wherein processor 1102 transmits candidate molecule data to remote molecular docking engines 1604, ADMET predictors, or database query tools through network 1110 and receives validation results through communication interface 1108. Said network connectivity enables distributed execution of the validation loop 1610 across multiple computing resources, potentially reducing latency for computationally intensive validation operations such as molecular dynamics simulations or protein-ligand docking calculations.

In some aspects, volatile memory 1104 stores intermediate computation results, model activations, and gradient values during training operations, while non-transitory memory 1106 stores trained model parameters, training checkpoints, and candidate molecule records as described with reference to the deployment step 924 of process 900 in FIG. 9. Operating system 1134 manages resource allocation across GPU 1128, AI 1130, and processor 1102 components, enabling concurrent execution of candidate molecule generation, feedback score computation, and parameter update operations during the training methodology described with reference to FIG. 6. In an embodiment, input 1112 receives natural language user prompts 1700 as described with reference to FIG. 17, said user prompts specifying drug design objectives, target biological properties, or structural constraints that configure execution of method 700 illustrated in FIG. 7. Output 1114 presents generated drug candidates 714, feedback scores, property distribution visualizations as described with reference to FIGS. 10A, 10B, and 10C, and validation results to users for review and selection of candidates for progression to in-vitro preparation 716 and experimental validation stages. Application software 1136 comprises program code implementing the drug-property evaluation model, generative chemistry language model, policy optimization algorithms, and tool-calling interfaces that collectively enable the multi-step automated drug discovery workflow described with reference to FIG. 17, said program code stored in non-transitory memory 1106 and executed by processor 1102 in coordination with GPU 1128 and AI 1130 accelerators.

FIGS. 12A, 12B, and 12C show examples of integrated language-molecule computing architectures, in accordance with an embodiment of the present disclosure, illustrating multiple modes of interaction between a language model (LM) and a language-conditioned molecular model (LCM). In one embodiment, corresponding to FIG. 12A, natural language input text containing molecular spans delimited by special tokens (e.g., <SOM> and <EOM>) is tokenized into embeddings (1212, 1214) and processed jointly with molecule-conditioned embeddings generated by a molecule encoder (1206), where fusion is achieved via cross-attention (1210) within a shared embedding layer (1208) and a unified LCM/LLM backbone (1200, 1202). In another embodiment, shown in FIG. 12B, the molecule encoder (1206) produces molecular embeddings that are projected into the language embedding space (1208) and provided as additional context to separate LCM (1200) and LLM (1202) models. In a further embodiment, depicted in FIG. 12C, the LLM (1202) performs reasoning and tool calls, invokes the LCM (1200), and receives a molecular-aware output (1216), enabling final text outputs or tool invocations (1218).

In some embodiments, the integrated language-molecule computing architectures illustrated in FIGS. 12A, 12B, and 12C operate as the core processing framework for the LLM-LCM 1600 described with reference to FIG. 16 and the LLM-LCM 1702 described with reference to FIG. 17, enabling unified processing of natural language instructions and molecular data within a single computational pipeline. Processor 102 executes the LCM 1200 and LLM 1202 components using the GPU 1128 and AI 1130 accelerators of computing architecture 1100 described with reference to FIG. 11, said accelerators providing parallel computation capabilities for transformer-based attention operations and embedding projections performed by the cross attention 1210 mechanism. In an aspect, the molecule encoder 1206 is trained during the supervised fine-tuning stage described with reference to FIGS. 9, 15A, and 15B, wherein processor 102 optimizes encoder parameters to produce molecular embeddings that capture chemical and structural features relevant to drug property prediction and candidate molecule generation. Said molecular embeddings produced by molecule encoder 1206 may encode information complementary to the sequential molecular representations described with reference to FIGS. 2A and 2B, including three-dimensional stereochemical configurations, electronic properties, or pharmacophoric feature arrangements not explicitly represented in SMILES or SELFIES token sequences. In some cases, the embedding layer 1208 maps token IDs 1214 to token embeddings 1212 using learned embedding matrices stored in memory 104, said embedding matrices initialized from pretrained language model weights and subsequently fine-tuned during the training methodology described with reference to FIG. 6. Processor 102 combines the token embeddings 1212 with projected molecular embeddings from molecule encoder 1206 through the cross attention 1210 mechanism, enabling the LLM 1202 to attend to molecular features when generating text output or final tool calls 1218.

In an embodiment, the LCM invocation response 1216 comprises intermediate representations produced by the LCM 1200 component that are consumed by the LLM 1202 component for subsequent natural language generation or reasoning operations. Said LCM invocation response 1216 may encode predicted molecular properties, structural assessments, or candidate molecule representations that inform the LLM 1202 when generating explanations, answering queries about molecular characteristics, or deciding whether to invoke external validation tools as described with reference to FIG. 16. In some aspects, the text output or final tool calls 1218 produced by the integrated architecture support the agentic capabilities described with reference to FIG. 16, wherein processor 102 generates tool call requests for molecular docking tools 1604, database query tools, or customized tools 1606 based on reasoning performed by the LLM 1202 component operating on molecular information provided by the LCM 1200 component. Said tool calls enable the integrated LLM-LCM system to autonomously gather additional information, validate candidate molecules, or refine molecular designs through the validation loop 1610 without requiring explicit user intervention for each validation step. In an embodiment, the architectures of FIGS. 12A, 12B, and 12C represent alternative integration configurations that may be selected based on computational resource availability, task requirements, or desired balance between molecular processing fidelity and natural language reasoning capability, said configurations stored as architecture templates in memory 104 and instantiated by processor 102 during initialization of the drug-like candidate compound design system 100.

FIGS. 13A and 13B show examples of language-molecule model integration architectures using token-wise and sequence-wise molecular injection, in accordance with an embodiment of the present disclosure. As illustrated in FIG. 13A, input text containing molecular spans delimited by special tokens (e.g., <SOM> and <EOM>) is tokenized into embedding vectors (1312, 1314) and processed by a shared embedding layer (1308), while a molecule encoder (1306) generates molecule-aware representations that are fused into the LCM/LLM backbone (1300, 1302) via cross-attention (1310), where query vectors from the language tokens attend over molecular key-value embeddings. In another embodiment, shown in FIG. 13B, molecular information from a database or list of molecules (1316) is encoded by the molecule encoder (1306) and injected into the LCM/LLM (1300, 1302) using cross-attention (1310) or projection, enabling sequence-wise conditioning for retrieval, screening, or reference. The integrated system produces a natural language response and molecule candidate design (1318), supporting both autoregressive generation and molecular reasoning.

In some embodiments, the language-molecule model integration architecture illustrated in FIG. 13A operates in conjunction with the multi-step automated drug discovery workflow described with reference to FIG. 17, wherein the LCM 1300 generates molecular embeddings 1314 for candidate molecules that are processed by the LLM 1302 to produce natural language responses and molecule candidate designs 1318 responsive to user prompts 1700. Processor 102 retrieves molecules from database or list of molecules 1316 stored in database 106 described with reference to FIG. 1, said molecules comprising reference compounds with known activity profiles, structural templates for scaffold-based design, or previously generated candidate molecules from earlier iterations of the molecule design phase 708. In an aspect, the molecule encoder 1306 processes each molecule retrieved from database 1316 to produce molecular embeddings 1314 that capture structural and chemical features relevant to the drug design task specified in the natural language input, said molecular embeddings projected into the embedding space of LLM 1302 through embedding layer 1308 for integration with text embeddings 1312 via cross attention 1310. Said cross attention mechanism enables the LLM 1302 to reason about relationships between multiple molecules simultaneously, supporting comparative analysis of candidate molecules, identification of structure-activity relationships, or selection of promising candidates from a population of generated molecules. In some cases, the NL response and molecule candidate design 1318 comprises both textual explanations describing predicted properties, structural features, or design rationale, and molecular representations specifying new or modified candidate molecules generated by the integrated system. Processor 102 parses said molecular representations from the NL response 1318 and converts them to sequential molecular representations as described with reference to FIGS. 2A and 2B for subsequent processing by the drug-property evaluation model of FIG. 3 and feedback score computation.

In an embodiment, the architecture of FIG. 13A supports virtual screening operations wherein processor 102 retrieves a plurality of molecules from database 1316, computes molecular embeddings 1314 for each retrieved molecule, and generates NL responses 1318 that rank or classify the retrieved molecules based on predicted suitability for the specified drug design objective. Said virtual screening may be invoked during the preparation phase 704 of FIG. 17 to identify starting points for de novo molecule generation or during the validation phase to compare generated candidate molecules against known active compounds. In some aspects, the sequence-wise injection of molecular embeddings 1314 illustrated in FIG. 13A enables processing of complete molecular representations without requiring token-by-token alignment between molecular and textual sequences, said sequence-wise injection supporting integration of molecular information from external sources including protein structure encodings, gene expression profiles, or pharmacophore models that inform target-specific candidate molecule generation. Processor 102 may further utilize the architecture of FIG. 13A to implement the helper tool calls 710 described with reference to FIG. 17, wherein the LLM 1302 generates natural language queries requesting molecular analysis, property prediction, or structural comparison, and the LCM 1300 processes said queries using molecular embeddings 1314 derived from candidate molecules under evaluation. Said helper tool calls support iterative refinement of candidate molecule designs during the molecule design phase 708, enabling the integrated system to reason about molecular modifications, assess predicted property changes, and generate improved candidate molecules through the refinement step 712 until drug candidates 714 satisfying predetermined criteria are identified.

FIG. 14 shows an example of graph-based molecular encoding and atom-wise embedding integration with a large chemistry model, in accordance with an embodiment of the present disclosure. As illustrated, a molecular representation (1402), such as a SMILES sequence, is first converted into a molecular graph using a cheminformatics toolkit (e.g., RDKit), where atoms and bonds form nodes and edges, respectively. This graph is processed by a graph encoder (1404), which may include a graph neural network, message-passing network, or graph transformer, to generate graph embeddings comprising vertex embeddings Eᵥ₁, Eᵥ₂, . . . , Eᵥₙ (1406) and optional edge embeddings Eₑ₁, Eₑ₂, . . . , Eₑₘ (1408). The vertex embeddings are then combined with incident edge embeddings through aggregation and projection operations to form atom-wise embeddings (1410), for example by concatenation and projection P(E_{i,k}). These atom-wise embeddings E₁, E₂, . . . , Eₙ (1412) are injected into the large chemistry model (LCM) (1414), enabling the model to leverage explicit molecular topology, atom connectivity, and bond relationships during downstream molecular analysis, property prediction, and generative tasks.

In some embodiments, the graph-based molecular encoding and atom-wise embedding integration illustrated in FIG. 14 operates in conjunction with the sequential molecular representations described with reference to FIGS. 2A and 2B to provide complementary structural information to the large chemistry model 1414. Processor 102 constructs the molecular graph 1402 from a SMILES or SELFIES sequential representation by parsing bond connectivity and atom identity information, said molecular graph comprising vertices representing atoms and edges representing covalent bonds with associated bond order and stereochemistry attributes. In an aspect, the graph encoder 1404 comprises a graph neural network architecture that iteratively updates vertex embedding 1406 and edge embedding 1408 through message passing operations, wherein each vertex aggregates information from neighboring vertices and edges to produce contextualized representations capturing local chemical environment and extended topological features. Said message passing operations may be executed using the GPU 1128 and AI 1130 accelerators of computing architecture 1100 described with reference to FIG. 11, enabling efficient parallel computation across all vertices and edges of the molecular graph 1402. In some cases, the graph encoder 1404 is pretrained on large molecular databases stored in database 106 using self-supervised learning objectives comprising graph reconstruction, masked atom prediction, or contrastive learning between augmented graph views, said pretraining establishing generalizable molecular representations prior to fine-tuning for specific drug design tasks as described with reference to FIG. 9. Processor 102 stores pretrained graph encoder parameters in memory 104 and loads said parameters during initialization of the drug-like candidate compound design system 100. In an embodiment, the atom-wise embedding 1410 produced by graph encoder 1404 is aligned with token positions in the sequential molecular representation, enabling token-wise injection as described with reference to FIGS. 12A, 12B, and 12C, wherein each token embedding is augmented with corresponding graph-derived structural information. Said alignment may be performed by mapping each atom in the molecular graph 1402 to its corresponding token or tokens in the SMILES or SELFIES sequence, accounting for multi-character atom symbols, ring closure tokens, and branch notation tokens that do not correspond directly to individual atoms.

In some aspects, the atom-wise embedding sequence 1412 is processed by the large chemistry model 1414 in combination with text embeddings to enable joint reasoning about molecular structure and natural language instructions as described with reference to FIGS. 12A, 12B, 12C, 13A, and 13B. The large chemistry model 1414 may comprise the integrated LLM-LCM architecture wherein cross attention mechanisms attend to atom-wise embeddings 1412 when generating candidate molecules or producing natural language responses describing molecular properties and design rationale. In an embodiment, the graph-based encoding of FIG. 14 supports enhanced prediction of properties that depend on three-dimensional molecular topology, including binding affinity predictions that consider spatial arrangement of pharmacophoric features, ADMET property predictions that depend on molecular surface characteristics, and synthetic feasibility assessments that evaluate bond accessibility and reaction site geometry. Processor 102 may selectively invoke graph-based encoding for candidate molecules that require detailed structural analysis during the validation loop 1610 of FIG. 16, while utilizing computationally lighter sequential representations for initial candidate generation and screening operations during the molecule design phase 708 of FIG. 17. Said selective invocation enables processor 102 to balance computational efficiency with structural analysis fidelity based on the stage of the drug discovery workflow and the complexity of the candidate molecules under evaluation.

FIGS. 15A and 15B show examples of a multi-specialist training, reinforcement learning, and self-distillation framework for a large chemistry model (LCM), in accordance with an embodiment of the present disclosure. As illustrated in FIG. 15A, a supervised fine-tuned LCM (1500) serves as a base model and is branched into multiple specialist LLM-LCM models (1502A-1502C), each trained on a respective task (Task 1, Task 2, . . . Task N) using reinforcement learning (RL-train) with task-specific objectives. The specialists generate responses that are evaluated and stored as question-answer-reward tuples (Q, A, R) (1506). These outputs are distilled via supervised fine-tuning into a main LCM (SFT) (1508), followed by general RL training (1510) to produce a main LCM generalist (1512). FIG. 15B illustrates an alternative embodiment in which preference-based training is applied using response-pair datasets (1508) instead of direct RL, enabling aggregation of specialist knowledge into a unified generalist model while improving cross-task generalization and optimization performance.

In some embodiments, the multi-specialist training, reinforcement learning, and self-distillation framework illustrated in FIG. 15A operates in coordination with the policy optimization framework described with reference to FIG. 5 and the training methodology described with reference to FIG. 6 to produce generalist models capable of designing candidate molecules across diverse biological targets and optimization objectives. Processor 102 trains each LLM-LCM specialist 1502A, 1502B, 1502C using target-specific experimental activity labels retrieved from database 106, wherein each specialist model is optimized for a respective biological target or category of related targets sharing similar binding site characteristics or mechanism of action. In an aspect, the SFT trained LCM 1500 provides a common initialization point for all specialist models, said initialization establishing baseline molecular generation capabilities and chemical language understanding prior to target-specific reinforcement learning optimization. Processor 102 stores the SFT trained LCM 1500 parameters in memory 104 and copies said parameters to initialize each specialist model before commencing RL-training with target-specific reward models derived from the drug-property evaluation model of FIG. 3. In some cases, the question-answer-reward tuples 1506 generated by specialist models 1502A, 1502B, 1502C comprise molecular design queries, generated candidate molecule responses, and associated feedback scores computed using target-specific evaluation criteria, said tuples stored in memory 104 as training data for the main LCM SFT 1508 distillation stage. Processor 102 aggregates question-answer-reward tuples 1506 from all specialist models to create a diverse training dataset that spans multiple biological targets and optimization objectives, enabling the main LCM generalist 1512 to learn from the collective expertise of the specialist models.

In an embodiment, the main LCM SFT 1508 stage trains the generalist model using supervised fine-tuning on the aggregated tuples, wherein processor 102 optimizes model parameters to reproduce high-reward candidate molecule responses generated by specialist models across diverse query types. Said supervised distillation transfers target-specific knowledge from specialist models to the generalist model without requiring the generalist to independently discover optimal molecular designs for each target through reinforcement learning exploration. In some aspects, the RL-training 1510 stage further refines the main LCM generalist 1512 using multi-objective reinforcement learning, wherein processor 102 applies policy optimization algorithms comprising proximal policy optimization (PPO), guided reward policy optimization (GRPO), or direct preference optimization (DPO) with reward signals derived from multiple drug-property evaluation models corresponding to different biological targets or property dimensions. Said multi-objective RL-training enables the generalist model to balance competing optimization objectives and generate candidate molecules that satisfy constraints across multiple property dimensions simultaneously. In an embodiment, the main LCM generalist 1512 is deployed as the LLM-LCM 1702 within the multi-step automated drug discovery workflow described with reference to FIG. 17, wherein the generalist model receives natural language user prompts 1700 specifying novel biological targets or multi-target optimization objectives and generates drug candidates 714 by leveraging knowledge distilled from specialist models trained on related targets. Processor 102 may further extend the specialist training framework by iteratively adding new specialist models for emerging biological targets, generating additional question-answer-reward tuples 1506, and performing incremental distillation updates to the main LCM generalist 1512 without requiring complete retraining, said incremental updates enabling continuous expansion of the generalist model's target coverage as new experimental activity data becomes available in database 106. In some cases, processor 102 evaluates generalist model performance against held-out test targets not represented by any specialist model during the testing step 916 of process 900 described with reference to FIG. 9, said evaluation assessing the generalist model's ability to generalize molecular design capabilities to novel biological targets based on knowledge transferred from related specialist models.

FIG. 16 shows an example of an iterative tool-augmented molecule validation and refinement workflow driven by an integrated LLM-LCM system, in accordance with an embodiment of the present disclosure. As illustrated, an LLM-LCM (1600) generates and evaluates candidate molecules by invoking a set of available tools (1602), including docking tools (1604) and one or more customized tools (1606), such as user-provided synthesis or analysis APIs. The designed molecule is validated by issuing tool calls, and predicted activity signals returned as tool call responses (1608) are fed back to the LLM-LCM (1600). The system follows a structured loop (1610) in which an initial drug candidate is generated, validated through automated tool execution, and refined based on validation outcomes and optional user feedback. This cycle may be repeated across multiple validation stages (Validation 2 through Validation T) to iteratively improve molecular quality. Upon satisfying desired potency, feasibility, and property criteria, the workflow outputs a final drug candidate design (1612), enabling closed-loop, model-driven molecular optimization with external computational validation.

In some embodiments, the iterative tool-augmented molecule validation and refinement workflow illustrated in FIG. 16 operates as a core component of the multi-step automated drug discovery workflow described with reference to FIG. 17, wherein the LLM-LCM 1600 corresponds to the LLM-LCM 1702 that processes natural language user prompts 1700 and generates drug candidates 714 through iterative design and validation cycles. Processor 102 executes the LLM-LCM 1600 using the integrated language-molecule computing architectures described with reference to earlier figures, wherein molecular embeddings produced by molecule encoders 1206, 1306, or graph encoder 1404 are combined with natural language embeddings to enable joint reasoning about candidate molecule properties and validation outcomes. In an aspect, the available tools 1602 are registered in memory 104 as tool definitions comprising tool identifiers, input parameter specifications, output format descriptions, and invocation endpoints, said tool definitions enabling the LLM-LCM 1600 to autonomously select and invoke appropriate tools based on the current state of the validation loop 1610 and the requirements of the drug design task. Processor 102 parses tool call requests generated by the LLM-LCM 1600, validates input parameters against tool specifications, and transmits invocation requests through the network and communication interface 1120 of computing architecture 1100 described with reference to FIG. 11 to external tool services or local tool implementations. In some cases, the docking tool 1604 comprises molecular docking engines that compute predicted binding poses and binding affinity scores for candidate molecules against target protein structures retrieved during the preparation phase 704 of FIG. 17, said binding affinity scores incorporated into feedback score computation to produce modified feedback scores reflecting both predicted properties from the drug-property evaluation model of FIG. 3 and structural validation outcomes from docking simulations. Processor 102 stores docking results in memory 104 in association with corresponding candidate molecule records, enabling the LLM-LCM 1600 to reason about binding pose quality, interaction patterns, and potential structural modifications that may improve binding affinity during subsequent iterations of the validation loop 1610.

In an embodiment, the customized tool 1606 comprises user-defined or task-specific validation tools including ADMET predictors, pharmacophore matching tools, synthetic feasibility analyzers, structural alert filters, or literature search tools that retrieve relevant publications describing structure-activity relationships for the target biological target. Said customized tools enable extension of the validation workflow to incorporate domain-specific validation criteria or proprietary prediction models without requiring modification of the core LLM-LCM 1600 architecture. In some aspects, the tool call response 1608 comprises structured data returned by invoked tools, said structured data parsed by processor 102 and converted to natural language descriptions or numerical features that are provided to the LLM-LCM 1600 as context for subsequent reasoning and candidate molecule refinement operations. The LLM-LCM 1600 interprets tool call responses 1608 to assess whether candidate molecules satisfy validation criteria, identify property deficiencies requiring structural modification, or determine that sufficient validation evidence supports progression of candidates to the final drug candidate design 1612 output. In an embodiment, the validation loop 1610 continues for a predetermined maximum number of iterations or until the LLM-LCM 1600 determines that candidate molecules satisfy all specified validation criteria, said termination conditions corresponding to stopping criteria, including identification of candidate molecules exceeding predetermined feedback score thresholds or satisfying a plurality of drug-property constraints. Processor 102 stores the final drug candidate design 1612 in memory 104 along with associated validation evidence, predicted property values, and design rationale generated by the LLM-LCM 1600, said stored information supporting subsequent selection of candidates for in-vitro preparation 716 and experimental validation as described with reference to FIG. 17. In some cases, processor 102 updates at least one of the drug-property evaluation model or the generative chemistry language model based on discrepancies between predicted properties and validation tool outcomes observed during the validation loop 1610, said updates improving prediction accuracy for subsequent drug design campaigns.

FIG. 17 shows an example of a multi-step automated drug discovery workflow executed by an integrated LLM-LCM system, in accordance with an embodiment of the present disclosure. The process begins when a user provides a natural language user prompt (1700), which is processed by the LLM-LCM (1702) to interpret the user intent and goals during a preparation phase (1704). In this phase, the LLM-LCM (1702) performs tool calls such as database or literature search (1706) to gather relevant background knowledge. The system then enters a molecule design phase (1708), where the LLM-LCM (1702) performs autoregressive generation to design one or more drug candidates (1714). During this phase, helper tool calls (1710), such as RDKit-based analysis, may be invoked. If a generated candidate is hard to synthesize or infeasible, the design is refined (1712) through additional autoregressive iterations. Promising candidates proceed to in-vitro preparation (1716), including automated synthesis planning and feasibility checks, followed by in-vitro validation and human feedback (1718). This iterative loop (1720) continues until a suitable drug candidate is obtained.

In some embodiments, the multi-step automated drug discovery workflow illustrated in FIG. 17 integrates the computational components described with reference to earlier figures into a unified pipeline that progresses from natural language task specification through experimental validation and iterative model refinement. Processor 102 receives the user prompt 1700 through input 1112 of computing architecture 1100 described with reference to FIG. 11, said user prompt specifying drug design objectives including target biological targets, desired property profiles, structural constraints, or multi-objective optimization criteria. The LLM-LCM 1702 parses the user prompt 1700 using natural language understanding capabilities of the LLM component and configures the LCM component for candidate molecule generation based on the specified objectives, said configuration comprising selection of appropriate specialist models from the multi-specialist framework of FIGS. 15A and 15B or activation of target-specific parameter sets within the main LCM generalist 1512. In an aspect, the preparation phase 1704 invokes database or literature search 1706 through tool calls to available tools 1602 described with reference to FIG. 16, wherein processor 102 retrieves known active compounds, target protein structures, binding site characteristics, and reported structure-activity relationships from database 106 or external literature databases. Said retrieved information is encoded using the molecular encoders and embedding layers described with reference to FIGS. 12A, 12B, 12C, 13A, 13B, and 14 and provided as context to the LLM-LCM 1702 for informed candidate molecule generation during the molecule design phase 1708.

In some cases, the molecule design phase 1708 generates candidate molecules using the generative chemistry language model trained through the policy optimization framework of FIG. 5 and training methodology of FIG. 6, wherein processor 102 produces sequential molecular representations as described with reference to FIGS. 2A and 2B and computes numerical chemical descriptors as described with reference to FIG. 4 for each generated candidate. Processor 102 evaluates generated candidates using the drug-property evaluation model of FIG. 3 to compute feedback scores, and the LLM-LCM 1702 invokes helper tool calls 1710 comprising RDKit-based property analysis, structural alert filtering, or synthetic feasibility scoring to assess candidate suitability. In an embodiment, the refinement step 1712 corresponds to iterative parameter updating wherein processor 102 updates parameters of the generative chemistry language model based on feedback scores and validation outcomes, generating subsequent candidate molecules having improved predicted properties until drug candidates 1714 satisfying predetermined stopping criteria are identified. Said refinement may incorporate external validation results from the validation loop 1610 of FIG. 16, wherein docking tool 1604 and customized tool 1606 outputs modify feedback scores to guide generation toward candidates exhibiting favorable binding characteristics and ADMET properties. In some aspects, the in-vitro preparation 1716 stage invokes automated synthesis planning tools to assess synthetic feasibility and generate retrosynthetic routes for promising drug candidates 1714, said synthesis planning supporting selection of candidates for experimental synthesis based on practical manufacturability considerations in addition to predicted biological activity.

Processor 102 stores synthesis plans in memory 104 in association with corresponding candidate molecule records, enabling chemist review and selection of candidates for laboratory synthesis. In an embodiment, the in-vitro validation and human feedback 1718 stage receives experimental assay results from synthesized candidates and explicit feedback from chemists reviewing candidate properties, predicted-versus-observed property comparisons, and structural characteristics. Processor 102 parses said experimental results and human feedback and updates at least one of the drug-property evaluation model or the generative chemistry language model, incorporating empirical outcome data into model parameters to improve prediction accuracy for subsequent iterations of the workflow. In some cases, the iterative loop 1720 returns to the preparation phase 1704 or molecule design phase 1708 with updated models and accumulated knowledge from prior iterations, enabling progressive refinement of drug candidates across multiple cycles until candidates suitable for progression to in-vivo validation and clinical evaluation are identified. Said closed-loop refinement enables the drug-like candidate compound design system 100 to continuously improve through integration of computational predictions, external validation outcomes, and experimental observations, potentially accelerating identification of therapeutically viable drug candidates relative to conventional drug discovery approaches that lack adaptive feedback integration.

### Additional Implementation Details

Although an example processing system has been described above, implementations of the subject matter and the functional operations described herein can be implemented in other types of digital electronic circuitry, or in computer software, firmware, or hardware, including the structures disclosed in this specification and their structural equivalents, or in combinations of one or more of them.

Embodiments of the subject matter and the operations described herein can be implemented in digital electronic circuitry, or in computer software, firmware, or hardware, including the structures disclosed in this specification and their structural equivalents, or in combinations of one or more of them. Embodiments of the subject matter described herein can be implemented as one or more computer programs, i.e., one or more modules of computer program instructions, encoded on computer storage medium for execution by, or to control the operation of, information/data processing apparatus. Alternatively, or in addition, the program instructions can be encoded on an artificially generated propagated signal, e.g., a machine-generated electrical, optical, or electromagnetic signal, which is generated to encode information/data for transmission to suitable receiver apparatus for execution by an information/data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The operations described herein can be implemented as operations performed by an information/data processing apparatus on information/data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various computing model infrastructures, such as web services, distributed computing, and grid computing infrastructures.

A computer program (also known as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A program can be stored in a portion of a file that holds other programs or information/data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described herein can be performed by one or more programmable processors executing one or more computer programs to perform actions by operating on input information/data and generating output. Processors suitable for the execution of a computer program include, by way of example, both general and special purpose microprocessors, and any one or more processors of any kind of digital computer. Generally, a processor will receive instructions and information/data from a read only memory or a random-access memory or both. The essential elements of a computer are a processor for performing actions in accordance with instructions and one or more memory devices for storing instructions and data. Generally, a computer will also include, or be operatively coupled to receive information/data from or transfer information/data to, or both, one or more mass storage devices for storing data, e.g., magnetic, magneto optical disks, or optical disks. However, a computer need not have such devices. Devices suitable for storing computer program instructions and information/data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, embodiments of the subject matter described herein can be implemented on a computer including a display device, e.g., a CRT (cathode ray tube) or LCD (liquid crystal display) monitor, for displaying information/data to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Embodiments of the subject matter described herein can be implemented in a computing system that includes a back end component, e.g., as an information/data server, or that includes a middleware component, e.g., an application server, or that includes a front end component, e.g., a client computer a graphical user interface or a web browser through which a user can interact with an implementation of the subject matter described herein, or any combination of one or more such back end, middleware, or front end components. The components of the system can be interconnected by any form or medium of digital information/data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some embodiments, a server transmits information/data (e.g., an HTML page) to a client device (e.g., for purposes of displaying information/data to and receiving user input from a user interacting with the client device). Information/data generated at the client device (e.g., a result of the user interaction) can be received from the client device at the server.

While this specification contains many specific implementation details, these should not be construed as limitations on the scope of any embodiment or of what may be claimed, but rather as descriptions of features specific to particular embodiments. Certain features that are described herein in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

In some embodiments of the present invention, the entire system can be implemented and offered to the end-users and operators over the Internet, in a so-called cloud implementation. No local installation of software or hardware would be needed, and the end-users and operators would be allowed access to the systems of the present invention directly over the Internet, using either a web browser or similar software on a client, which client could be a desktop, laptop, mobile device, and so on. This eliminates any need for custom software installation on the client side and increases the flexibility of delivery of the service (software-as-a-service), and increases user satisfaction and ease of use. Various business models, revenue models, and delivery mechanisms for the present invention are envisioned, and are all to be considered within the scope of the present invention.

In general, the method executed to implement the embodiments of the invention, may be implemented as part of an operating system or a specific application, component, program, object, module or sequence of instructions referred to as “computer program(s)” or “computer code(s).” The computer programs typically comprise one or more instructions set at various times in various memory and storage devices in a computer, and that, when read and executed by one or more processors in a computer, cause the computer to perform operations necessary to execute elements involving the various aspects of the invention. Moreover, while the invention has been described in the context of fully functioning computers and computer systems, those skilled in the art will appreciate that the various embodiments of the invention are capable of being distributed as a program product in a variety of forms, and that the invention applies equally regardless of the type of machine or computer-readable media used to affect the distribution. Examples of computer-readable media include but are not limited to recordable type media such as volatile and non-volatile (or non-transitory) memory devices, floppy and other removable disks, hard disk drives, optical disks, which include Compact Disk Read-Only Memory (CD ROMs), Digital Versatile Disks (DVDs), etc., as well as digital and analog communication media.

### CONCLUSIONS

One of ordinary skill in the art knows that the use cases, structures, schematics, and flow diagrams may be performed in other orders or combinations, but the inventive concept of the present invention remains without departing from the broader scope of the invention. Every embodiment may be unique, and methods/steps may be either shortened or lengthened, overlapped with the other activities, postponed, delayed, and continued after a time gap, such that every use case and application is accommodated to practice the methods of the present invention.

Although the present invention has been described with reference to specific exemplary embodiments, it will be evident that the various modifications and changes can be made to these embodiments without departing from the broader scope of the invention. Accordingly, the specification and drawings are to be regarded in an illustrative sense rather than in a restrictive sense. It will also be apparent to the skilled artisan that the embodiments described above are specific examples of a single broader invention which may have greater scope than any of the singular descriptions taught. There may be many alterations made in the descriptions without departing from the scope of the present invention.

For simplicity of explanation, the embodiments of the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. Reference throughout this specification to “an embodiment,” “certain embodiments,” or “one embodiment” means that a particular feature, structure, or characteristic described in connection with the embodiment is included in at least one embodiment. Thus, the appearances of the phrase “an embodiment,” “certain embodiments,” or “one embodiment” in various places throughout this specification are not necessarily all referring to the same embodiment.

What is claimed is:

1. A system to design a drug-like candidate compound, comprising:
a processor; and
a memory coupled to the processor, the memory storing program code, the program code executable by the processor, the program code executable by the processor, the program code comprising code to:
retrieve, from a database, a plurality of molecular training data comprising molecular structure information for a plurality of training molecules and a plurality of drug-property matrices associated with the plurality of training molecules;
generate, for each training molecule in the plurality of training molecules, a sequential molecular representation selected from the group consisting of a plurality of Simplified Molecular Input Line Entry System (SMILES) strings and a plurality of Self-Referencing Embedded Strings (SELFIES) to form a plurality of sequential molecular representations;
compute a numerical chemical descriptor or chemical property associated with each training molecule in the plurality of training molecules to form a plurality of numerical chemical descriptors;
train a drug-property evaluation model using the plurality of sequential molecular representations and the plurality of numerical chemical descriptors, wherein the drug-property evaluation model is trained to output a feedback score indicative of at least one predicted drug property associated with the plurality of drug-property matrices;
generate, using a generative chemistry language model, at least one new candidate molecule to form a plurality of new candidate molecules;
compute, by the drug-property evaluation model, a feedback score for each new candidate molecule in the plurality of new candidate molecules to form a plurality of feedback scores; and
update one or more parameters of the generative chemistry language model based on the plurality of feedback scores, wherein updating the one or more parameters causes the generative chemistry language model to generate subsequent candidate molecules to form a plurality of subsequent candidate molecules, each having an improved feedback score relative to the plurality of new candidate molecules, and wherein updating the one or more parameters and generation of subsequent candidate molecules continues until at least one stopping criterion is satisfied.

2. The system of claim 1, wherein the plurality of drug-property matrices comprises at least one experimental activity label that is target-specific for at least one biological target, and wherein the at least one experimental activity label is selected from the group consisting of a binding affinity, a half maximal inhibitory concentration, a half maximal effective concentration, an inhibition constant, a dissociation constant, and an inhibition percentage.

3. The system of claim 1, wherein training the drug-property evaluation model comprises training a quantitative structure-property relationship (QSPR) predictor that maps the plurality of sequential molecular representations and the plurality of numerical chemical descriptors to the plurality of feedback scores.

4. The system of claim 1, wherein the plurality of numerical chemical descriptors comprises at least one selected from the group consisting of a physicochemical descriptor, a structural descriptor, a topological descriptor, a molecular fingerprint, a fragment descriptor, and a learned descriptor embedding.

5. The system of claim 1, wherein each sequential molecular representation in the plurality of sequential molecular representations further comprises information selected from the group consisting of an International Chemical Identifier (InChI) string, a molecular graph representation, a fingerprint vector, a substructure presence vector, a pharmacophore feature vector, and a scaffold representation.

6. The system of claim 1, wherein the feedback score comprises a multi-objective score comprising a potency component and a developability component, wherein the developability component comprises at least one selected from the group consisting of a selectivity factor, a stability factor, a novelty score, a synthesizability score, a similarity score, a potency score, an absorption score, a distribution score, a metabolism score, a toxicity risk score, a solubility score, a permeability score, and a clearance score.

7. The system of claim 6, wherein the multi-objective score is computed using a weighted aggregation of a plurality of objective-specific subscores and one or more threshold-based filters that reject at least one new candidate molecule or at least one subsequent candidate molecule that violates a predetermined constraint.

8. The system of claim 1, wherein the at least one stopping criterion comprises at least one selected from the group consisting of:
identification of at least one subsequent candidate molecule with an associated feedback score exceeding a predetermined threshold;
identification of at least one subsequent candidate molecule satisfying a plurality of drug-property constraints;
reaching of a maximum number of update iterations;
convergence of the plurality of feedback scores across the plurality of new candidate molecules;
meeting of a diversity criterion across a subset of the plurality of new candidate molecules; and
selection of a ranked set of candidate molecules from the plurality of new candidate molecules satisfying a selection rule.

9. The system of claim 1, wherein the program code to update the one or more parameters of the generative chemistry language model comprises applying a policy optimization algorithm, wherein the policy optimization algorithm comprises at least one process selected from the group consisting of a proximal policy optimization (PPO), a guided reward policy optimization (GRPO), and a direct preference optimization (DPO).

10. The system of claim 9, wherein the program code to update the one or more parameters further comprises applying Kullback-Leibler (KL) regularization relative to a reference policy to constrain divergence between an updated policy and the reference policy.

11. The system of claim 1, wherein the program code further comprises code to:
validate, each new candidate molecule in the plurality of new candidate molecules or each subsequent candidate molecule in the plurality of subsequent candidate molecules using one or more external in-silico validation tools, wherein the one or more external in-silico validation tools comprise at least one tool selected from the group consisting of a molecular docking engine, a molecular dynamics or simulation engine, an ADMET (absorption, distribution, metabolism, excretion, and toxicity) predictor, a pharmacophore predictor, a cheminformatics toolkit, a database query tool, and a literature search tool; and
modify the feedback score of each new candidate molecule in the plurality of new candidate molecules or each subsequent candidate molecule in the plurality of subsequent candidate molecules to produce a modified feedback score for each validated new candidate molecule or each validated subsequent candidate molecule.

12. The system of claim 11, wherein the processor updates the one or more parameters of the generative chemistry language model based on the modified feedback scores that causes the generative chemistry language model to generate a plurality of further refined candidate molecules.

13. The system of claim 1, wherein the processor trains a target-specific or task-specific specialist generative chemistry language model using a plurality of target-specific or task-specific experimental activity labels, wherein the target-specific specialist generative chemistry language model is trained for a respective biological target, wherein the target-specific or task-specific experimental activity labels are associated with the respective biological target, to generate a plurality of target-specific candidate molecules.

14. The system of claim 1, wherein the processor further executes an integrated large language model (LLM) and large chemistry model (LCM) system that iteratively refines candidate molecules through a combined score-based and language-conditioned feedback loop, wherein:
the processor invokes, through an agentic tool-calling interface, one or more external in-silico validation tools comprising at least one of a molecular docking engine, an ADMET predictor, and a cheminformatics toolkit, and compute a score-based validation result for each candidate molecule based on outputs from the one or more external in-silico validation tools and predicted drug property values from the drug-property evaluation model;
the processor receives natural language feedback from a human chemist, said natural language feedback comprising at least one of structural modification guidance, property preference indications, design rationale assessments, and predicted-versus-observed property evaluations for one or more candidate molecules; and
the processor updates at least one of the drug-property evaluation model and the generative chemistry language model based on a combination of the score-based validation result and the natural language feedback, wherein the natural language feedback is processed by the integrated LLM and LCM system to condition subsequent candidate molecule generation, and wherein the iterative refinement continues across multiple cycles of candidate molecule generation, score-based validation, and natural language feedback incorporation until candidate molecules satisfying predetermined drug design criteria are identified.

15. The system of claim 1, wherein the program code further comprises code to:
apply a plurality of structural alert filters to each new candidate molecule and each subsequent candidate molecule, wherein each structural alert filter in the plurality of structural alert filters comprises at least one of a reactive functional group filter, a toxicophore filter, a mutagenicity alert filter, Lipinski Rule-of-Five filter, metal chelator filter, unstable moiety filter, strained ring filter, genotoxicity alert filter, and a covalent-binding alert filter;
exclude each new candidate molecule and each subsequent candidate molecule whenever at least a pre-set number of the plurality of structural alert filters are triggered, to generate a plurality of excluded molecules; and
compute a plurality of non-excluded feedback scores for each new candidate molecule and each subsequent candidate molecule that is not in the plurality of excluded molecules.

16. A computer-executable method for designing a drug-like candidate compound, the computer-executable method comprising:
retrieving, from a database, a plurality of molecular training data comprising molecular structure information for a plurality of training molecules and a plurality of drug-property matrices associated with the plurality of training molecules;
generating, for each training molecule in the plurality of training molecules, a sequential molecular representation selected from the group consisting of a plurality of Simplified Molecular Input Line Entry System (SMILES) strings and a plurality of Self-Referencing Embedded Strings (SELFIES) to form a plurality of sequential molecular representations;
computing a numerical chemical descriptor or chemical property associated with each training molecule in the plurality of training molecules to form a plurality of numerical chemical descriptors;
training a drug-property evaluation model using the plurality of sequential molecular representations and the plurality of numerical chemical descriptors, wherein the drug-property evaluation model is trained to output a feedback score indicative of at least one predicted drug property associated with the plurality of drug-property matrices;
generating, using a generative chemistry language model, at least one new candidate molecule to form a plurality of new candidate molecules;
computing, by the drug-property evaluation model, a feedback score for each new candidate molecule in the plurality of new candidate molecules to form a plurality of feedback scores; and
updating one or more parameters of the generative chemistry language model based on the plurality of feedback scores, wherein updating the one or more parameters causes the generative chemistry language model to generate subsequent candidate molecules to form a plurality of subsequent candidate molecules, each having an improved feedback score relative to the plurality of new candidate molecules, and wherein updating the one or more parameters and generation of subsequent candidate molecules continues until at least one stopping criterion is satisfied.

17. The computer-executable method of claim 16, further comprising:
computing a synthetic feasibility score for each new candidate molecule in the plurality of new candidate molecules or each subsequent candidate molecule in the plurality of subsequent candidate molecules and updating the corresponding feedback score of each new candidate molecule or each subsequent candidate molecule in response to the synthetic feasibility score.

18. The computer-executable method of claim 17, further comprising, upon satisfaction of the at least one stopping criterion:
selecting at least one subsequent candidate molecule for synthesis with an associated synthetic feasibility score that exceeds a synthetic feasibility score threshold.

19. The computer-executable method of claim 18, further comprising:
updating at least one of the drug-property evaluation model and the generative chemistry language model based on an experimental assay result of the synthesized at least one subsequent candidate molecule.

20. A non-transitory computer-readable storage medium storing program code, the program code executable by a hardware processor, the program code when executed by the hardware processor causing the hardware processor to execute a computer-implemented method for designing a drug-like candidate compound, the program code comprising code to:
retrieve, from a database, a plurality of molecular training data comprising molecular structure information for a plurality of training molecules and a plurality of drug-property matrices associated with the plurality of training molecules;
generate, for each training molecule in the plurality of training molecules, a sequential molecular representation selected from the group consisting of a plurality of Simplified Molecular Input Line Entry System (SMILES) strings and a plurality of Self-Referencing Embedded Strings (SELFIES) to form a plurality of sequential molecular representations;
compute a numerical chemical descriptor associated with each training molecule in the plurality of training molecules to form a plurality of numerical chemical descriptors;
train a drug-property evaluation model using the plurality of sequential molecular representations and the plurality of numerical chemical descriptors, wherein the drug-property evaluation model is trained to output a feedback score indicative of at least one predicted drug property associated with the plurality of drug-property matrices;
generate, using a generative chemistry language model, at least one new candidate molecule to form a plurality of new candidate molecules;
compute, by the drug-property evaluation model, a feedback score for each new candidate molecule in the plurality of new candidate molecules to form a plurality of feedback scores; and
update one or more parameters of the generative chemistry language model based on the plurality of feedback scores, wherein updating the one or more parameters causes the generative chemistry language model to generate subsequent candidate molecules to form a plurality of subsequent candidate molecules, each having an improved feedback score relative to the plurality of new candidate molecules, and wherein updating the one or more parameters and generation of subsequent candidate molecules continues until at least one stopping criterion is satisfied.
